(19) United States
(12) Patent Application Publication
(10) Pub. No.: US 2018/0179282 A1
(43) Pub. Date: Jun. 28, 2018

(54) TREATMENT OF CANCER BY COMBINED BLOCKADE OF THE PD-1 AND CXCR4 SIGNALING PATHWAYS

(71) Applicant: BRISTOL-MYERS SQUIBB COMPANY, Princeton, NJ (US)

(72) Inventors: Josephine M. Cardarelli, San Carlos, CA (US); Wendy L. Clemens, Newtown, PA (US); Glenn S. Kroog, New York, NY (US); Daniel E. Lopes de Menezes, Berkeley, CA (US); Chin Pan, Los Altos, CA (US); Paul D. Ponath, San Francisco, CA (US); Jean Viallet, Malvern, PA (US)

(21) Appl. No.: 15/735,055

(22) PCT Filed: Jun. 13, 2016
(86) PCT No.: PCT/US2016/037207

§ 371 Date: Dec. 8, 2017

Related U.S. Application Data
(60) Provisional application No. 62/174,931, filed on Jun. 12, 2015.

(51) Int. Cl. C07K 16/28; A61P 35/00; C07K 16/24

(52) U.S. Cl. CPC C07K 16/2818; A61P 35/00; C07K 16/2827; A61K 2039/507; C07K 16/24; C07K 2317/76; C07K 2317/21; C07K 16/2866

(57) ABSTRACT

This disclosure provides a method for treating a subject afflicted with a cancer comprising administering to the subject a combination of therapeutically effective amounts of an antibody or an antigen-binding portion thereof that binds specifically to Programmed Death-1 (PD-1) or to Programmed Death Ligand-1 (PD-L1), and an antibody or an antigen-binding portion thereof that binds specifically to C-X-C Chemokine Receptor 4 (CXCR4) or to C-X-C motif chemokine 12 (CXCL12). The disclosure also provides a kit for treating a subject afflicted with a cancer, the kit comprising one or more dosages of an antibody or an antigen-binding portion thereof that binds specifically to PD-1 or to PD-L1, one or more dosages of an antibody or an antigen-binding portion thereof that binds specifically to CXCR4 or to CXCL12, and instructions for using the antibodies or portions thereof for treating the subject.

### CROSS REFERENCE TO RELATED APPLICATIONS

This application is entitled to priority pursuant to 35 U.S.C. § 119(e) to U.S. Provisional Application No. 62/174,931, filed Jun. 12, 2015, which is incorporated herein in its entirety.

Throughout this application, various publications are referenced in parentheses by author name and date, or by Patent No. or Patent Publication No. Full citations for these publications may be found at the end of the specification immediately preceding the claims. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

### FIELD OF THE INVENTION

This invention relates to methods for treating a cancer in a subject comprising administering to the subject a combination of an antibody that blocks the Programmed Death-1 (PD-1)/Programmed Death Ligand-1 (PD-L1) signaling pathway and an antibody that blocks the C-X-C Chemokine Receptor 4 (CXCR4)/C-X-C motif chemokine 12 (CXCL12) signaling pathway.

### BACKGROUND OF THE INVENTION

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system makes immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have provided new immunotherapeutic approaches for treating cancer, including the development of an antibody (Ab), ipilimumab (YERVOY®), that binds to and inhibits Cytotoxic T-Lymphocyte Antigen-4 (CTLA-4) for the treatment of patients with advanced melanoma (Hodi et al., 2010) and the development of Abs such as nivolumab (OPDIVO®) and pembrolizumab (KEYTRUDA®) that bind specifically to the PD-1 receptor, a cell surface negative regulatory molecule expressed by activated T and B lymphocytes, and block the inhibitory PD-1/PD-1 ligand pathway (Topalian et al., 2012a, b; Topalian et al., 2014; Hamid et al., 2013; Hamid and Carvajal, 2013; McDermott and Atkins, 2013). This pathway can also be disrupted by Abs that bind specifically to PD-L1, including BMS-936559 (PCT Publication No. WO 2013/173223) and atezolizumab (TECENTRIQ®; Fehrenbacher et al., 2016).

Nivolumab (previously designated BMS-936558, MDX-1106, or ONO-4538, and designated 5C4 in U.S. Pat. No. 8,008,449) is a fully human immunoglobulin (Ig) G4 (S228P) monoclonal antibody (mAb) that selectively prevents interaction with the PD-1 ligands, PD-L1 and PD-L2 (U.S. Pat. No. 8,008,449; Wang et al., 2014), thereby blocking the down-regulation of antigen-specific T cell responses directed against both foreign (including tumor) and self antigens and enhancing an immune response against these antigens (McDermott and Atkins, 2013). Nivolumab has received approval recently for metastatic melanoma, squamous non-small cell lung cancer (NSCLC), renal cell carcinoma (RCC) and classical H-odgkin lymnphoma (cHL), and is currently being clinically evaluated as monotherapy or in combination with ipilimumab or other anti-cancer agents for efficacy in various tumor types, including pancreatic cancer (PAC), small cell lung cancer (SCLC), head and neck cancer, bladder cancer and hematological malignancies (see, e.g., Topalian et al., 2012b; WO 2013/173223; Ansell et al., 2015; and NCT02309177, NCT01928394, NCT02105636, NCT02387996, and NCT02329847 on the Clinical Trials Website, http://www.clinicaltrials.gov). However, combinations of nivolumab with other targeted therapies may further improve response rates and prolong survival in a higher percentage of patients. Specifically, for example, the combination of nivolumab with therapies targeting the protective stromal microenvironment surrounding the tumor may allow for enhanced infiltration of activated immune cells to the tumor site, thereby increasing tumor cell killing and broadening the spectrum of patients able to benefit from these therapies.

Ulocuplumab (previously designated BMS-936564 or MDX-1338, and designated F7 in WO 2008/060367) is a fully human IgG4 (S224P) mAb specific for CXCR4, which is expressed on leukocytes, platelets and other non-hematopoietic cells that comprise the tumor stromal microenvironment (Balkwill, 2004). CXCR4 is also over-expressed in the majority of human cancers and, together with its endogenous ligand CXCL12, plays a fundamental role in cancer pathogenesis including proliferation, adhesion, metastasis, angiogenesis and survival (Domanska et al., 2013; Duda et al., 2011; Balkwill, 2004; Pitt et al., 2015; Passoro et al., 2015; WO 2008/060367). Ulocuplumab has been evaluated in two Phase 1 clinical trials in subjects with various hematological malignancies including acute myeloid leukemia (AML), multiple myeloma (MM), chronic lymphocytic leukemia (CLL), follicular lymphoma (FL) and diffuse large B cell lymphoma (DLBCL) with a safe and tolerable profile. Efficacy data from the AML and MM cohorts has been presented and show encouraging results for the addition of ulocuplumab to standard therapy (Becker et al., 2014; Ghobrial et al., 2014).

Evidence has been presented suggesting that CXCL12 may be immunosuppressive and may support the stroma surrounding the tumor, shielding it from immune mechanisms that would otherwise result in tumor cell killing (Domanska et al., 2013; Duda et al., 2011; Burger and Kipps, 2006). The refractory nature of many metastatic tumors, including PAC and SCLC, may result from an immunosuppressive environment surrounding the tumor that prevents activated lymphocytes from accessing the tumor site. It is, therefore, of interest to determine whether disruption of the stromal microenvironment via CXCR4 blockade with an anti-CXCR4 Ab could increase the tumor's susceptibility to immune-targeted therapies and allow for the penetration of immune cells to the tumor site. Furthermore, ulocuplumab may be involved in direct cytotoxicity against the tumor since it has demonstrated direct in vitro cell killing activity of CXCR4-expressing tumor cells (Kuhne et al., 2013; WO 2013/071068). CXCR4 is also over-expressed on immune-suppressive regulatory T cells (Tregs) and myeloid-derived suppressor cells (MDSCs) in cancer patients (Wang et al., 2012; Obermajer et al., 2011; Katoh and Watanabe, 2015), and anti-CXCR4-mediated depletion of Tregs and/or MDSCs may contribute to enhancement of an anti-tumor effect.

The present disclosure relates to studies evaluating Ab-mediated dual blockade of the PD-1/PD-L1 and CXCR4/CXCL12 signaling pathways to determine whether the combined inhibition of these pathways benefit cancers that are poorly treated by standard therapies. The combination of the mechanisms of action of anti-CXCR4/anti-CXCL12 and anti-PD-1/anti-PD-L1 offers a unique opportunity to simultaneously target the immunosuppressive tumor microenvironment and the activation of T cells, thus increasing tumor cell killing.

### SUMMARY OF THE INVENTION

The present disclosure provides a method for treating a subject afflicted with a cancer comprising administering to the subject a combination of therapeutically effective amounts of: (a) an Ab or an antigen-binding portion thereof that binds specifically to PD-1 or to PD-L1; and (b) an Ab or an antigen-binding portion thereof that binds specifically to CXCR4 or to CXCL2. In certain embodiments, the Ab that binds specifically to PD-1 or to PD-L1 disrupts the interaction between PD-1 and PD-L1, and inhibits PD-1/PD-L1 signaling. In other embodiments, the Ab that binds to CXCR4 or CXCL2 disrupts the interaction between CXCR4 and CXCL12, and inhibits CXCR4/CXCL12 signaling. In further embodiments, the cancer is a solid tumor such as PAC, SCLC or hepatocellular carcinoma (HCC). In certain embodiments of any of the therapeutic methods disclosed herein, the Ab that binds to PD-1 is nivolumab or pembrolizumab. In certain other embodiments, the Ab that binds specifically to PD-L1 is BMS-936559, atezolizumab, durvalumab, STI-A1014 or avelumab. In yet other embodiments, the Ab that the Ab that binds specifically to CXCR4 is ulocuplumab, or preferably, ulocuplumab modified to comprise an Fc region with effector functions, for example an Fc region of a human IgG1 or human IgG3 isotype. In further embodiments, the Ab that binds specifically to CXCL2 is the mAb designated 2A5 in U.S. Pat. No. 8,496,931.

In certain embodiments of the methods comprising use of an anti-PD-1 Ab in combination with an anti-CXCR4 Ab, the therapeutically effective dosage of the anti-PD-1 Ab or antigen-binding portion thereof ranges from about 0.1 to about 20 mg/kg body weight administered by intravenous infusion about once every 2, 3 or 4 weeks. In certain preferred embodiments, the anti-PD-1 Ab is administered at a dose of about 2 mg/kg or about 3 mg/kg once every 2 or 3 weeks. In certain other embodiments of these methods the therapeutically effective dosage of the anti-CXCR4 Ab or antigen-binding portion thereof ranges from a flat dose of about 50 to about 2000 mg administered weekly by intravenous infusion. In certain preferred embodiments, the anti-CXCR4 Ab is administered at a flat dose of about 400 or about 800 mg weekly.

The disclosure also provides a kit for treating a subject afflicted with a cancer, the kit comprising: (a) one or more dosages ranging from about 0.1 to about 20 mg/kg body weight of an Ab or an antigen-binding portion thereof that binds specifically to PD-1 or to PD-L1; (b) one or more dosages ranging from about 50 to about 2000 mg of an Ab or an antigen-binding portion thereof that binds specifically to CXCR4 or to CXCL12; and (c) instructions for using the Ab or portion thereof that binds specifically to PD-1 or to PD-L1 and the Ab or portion thereof that binds specifically to CXCR4 or to CXCL12.

Other features and advantages of the instant invention will be apparent from the following detailed description and examples which should not be construed as limiting. The contents of all cited references, including scientific articles, GenBank entries, patents and patent applications cited throughout this application are expressly incorporated herein by reference.

### BRIEF DESCRIPTION OF THE FIGURES

FIG. 1 shows an assessment of CXCR4 expression on mouse Kp1 and Kp3 SCLC cell lines by flow cytometry.

FIG. 2 shows an assessment of CXCR4 expression on the MC38 mouse colon adenocarcinoma cell line by flow cytometry.

FIG. 3 shows an assessment of CXCR4 expression on CD8+ T cells, T effector cells and regulatory T cells (Tregs) by flow cytometry.

FIG. 4 shows the effects on tumor growth of anti-mCXCR4 and anti-mouse PD-1 Abs used alone or in combination in a syngeneic endogenous CXCR4-expressing mouse SCLP model derived from a KP1 tumor cell line (p53; Rb1; p130 null; B6129S1/J F1 mice). A, Median change in tumor volume from treatment with single Abs compared to controls. B, Median change in tumor volume from treatment with combination of Abs compared to controls. Vehicle: saline; KLH mIgG1 (or mIgG1 KLH): anti-Keyhole Limpet Hemocyanin (KLH) mAb having mouse IgG1 isotype; mIgG2a KLH: anti-KLH mAb having mouse IgG2a isotype; mCXCR4 mIgG1 (4.8): anti-mouse CXCR4 Ab (clone 4.8) having mouse IgG1 isotype; mCXCR4 mIgG2a: anti-mouse CXCR4 Ab (clone 4.8) having mouse IgG2a isotype; mPD-1 mIgG1 (or simply “PD-1”): anti-PD-1 mAb 4H2 having mouse IgG1 isotype. Similar abbreviations are used in the other figures relating to anti-tumor efficacy studies in mouse tumor models.

FIG. 5 shows the effects on tumor growth of anti-mCXCR4 IgG2a and anti-mouse PD-1 Abs used alone or in combination in a syngeneic endogenous CXCR4-nonexpressing mouse SCLP model derived from a Kp3 tumor cell line (P53; Rb1; p130 null; B6129S1/J F1 mice). A, Median change in tumor volume from treatment with single Abs compared to controls. B, Median change in tumor volume from treatment with combination of Abs compared to controls.

FIG. 6 shows the effects of anti-mCXCR4 and anti-mouse PD-1 Abs used alone or in combination in a CXCR4-nonexpressing mouse colon carcinoma model derived from a MC38 tumor cell line (BC57BI/6 mice). A, Median change in tumor volume from treatment with single Abs compared to controls. B, Median change in tumor volume from treatment with combination of Abs compared to controls.

FIG. 7 shows the effects of the combination of anti-mCXCR4 mIgG2a and anti-mPD-1 mIgG1D265A Abs in combination in inhibiting the growth of a CXCR4-nonexpressing H22 liver cancer mouse model. A, Change in tumor volume in eight individual mice from treatment with anti-mCXCR4 plus anti-mPD-1. B, Change in tumor volume in eight individual mice from treatment with anti-mPD-1. C, Change in tumor volume in eight individual mice from treatment with combination of isotype controls. D, Median changes in tumor volumes for the treatments shown in (A) to (C).

FIG. 8 shows a schematic summarizing the design of a Phase 1/2 study of ulocuplumab in combination with nivolumab to evaluate the safety and efficacy of this combination of therapeutic Abs in subjects with SCLC and PAC.

FIG. 9 shows the receptor occupancy (RO) on circulating CD3⁺ cells (T cells) in the patient cohort dosed with a combination of 200 mg ulocuplumab weekly and 3 mg/kg nivolumab every two weeks. Data are depicted as absolute % RO by ulocuplumab on circulating CD3⁺ cells. Gray horizontal lines indicate median values. Each dot represents a subject sample.

### DETAILED DESCRIPTION OF THE INVENTION

The present invention relates to methods for treating solid tumors in a subject comprising administering a combination of an anti-PD-1 or an anti-PD-L1 Ab and an anti-CXCR4 or anti-CXCL12 Ab to the subject.

### Terms

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. A preferred route for administration of therapeutic Abs such as anti-PD-1 and anti-CXCR4 Abs is intravenous administration. Other routes of administration include intramuscular, subcutaneous, intraperitoneal, or other parenteral routes of administration, for example by injection or infusion. The phrase “parenteral administration” as used herein means modes of administration other than enteral and topical administration. Administering can also be performed, for example, once, a plurality of times, and/or over one or more extended periods.

An “adverse event” (AE) is any new untoward medical occurrence or worsening of a preexisting medical condition in a clinical investigation subject administered study drug and need not have a causal relationship with this treatment. An AE can therefore be any unfavorable and unintended sign (such as an abnormal laboratory finding), symptom, or disease temporally associated with the use of study drug, whether or not considered related to the study drug. The causal relationship to study drug is determined by a physician and is used to assess all AEs. The causal relationship can either “related” (i.e., there is a reasonable causal relationship between study drug administration and the AE), or “not related” (i.e., there is not a reasonable causal relationship between study drug administration and the AE). The term “reasonable causal relationship” means there is evidence to suggest a causal relationship. Reference to methods or dosages for “reducing adverse events” means a treatment regime, e.g., a combination of an anti-PD-1/anti-PD-L1 Ab and an anti-CXCR4/anti-CXCL12 Ab, that decreases the incidence and/or severity of one or more AEs associated with the use of a different treatment regime, e.g., monotherapy with an anti-PD-1/anti-PD-L1 or an anti-CXCR4/anti-CXCL12 Ab.

A “serious adverse event” (SAE) is any untoward medical occurrence that at any dose results in death, is life-threatening (defined as an event in which the subject was at risk of death at the time of the event; it does not refer to an event which hypothetically might have caused death if it were more severe), requires inpatient hospitalization or causes prolongation of existing hospitalization, results in persistent or significant disability/incapacity, is a congenital anomaly/birth defect, and/or is an important medical event (defined as a medical event(s) that may not be immediately life-threatening or result in death or hospitalization but, based upon appropriate medical and scientific judgment, may jeopardize the subject or may require intervention to prevent a more serious outcome). Examples of such important medical events include, but are not limited to, intensive treatment in an emergency room or at home for allergic bronchospasm, blood dyscrasias or convulsions that do not result in hospitalization, and potential drug-induced liver injury (DILI).

An “antibody” (Ab) shall include, without limitation, a glycoprotein immunoglobulin (Ig) which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region of an IgG Ab comprises three constant domains, C_{H1}, C_{H2} and C_{H3}. Each light chain comprises a light chain variable region (abbreviated herein as V_{L}) and a light chain constant region. The light chain constant region of an IgG Ab comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1-CDR1-FR2-CDR2-FR3-CDR3-FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C q) of the classical complement system.

An Ig may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM, IgG1, or IgG4) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized partially or fully by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “isolated” Ab refers to an Ab that is substantially free of other Abs having different antigenic specificities (e.g., an isolated Ab that binds specifically to PD-1 is substantially free of Abs that bind specifically to antigens other than PD-1). An isolated Ab that binds specifically to PD-1 may, however, have cross-reactivity to other antigens, such as PD-1 molecules from different species. Moreover, an isolated Ab may be purified so as to be substantially free of other cellular material and/or chemicals.

The term “monoclonal” Ab (mAb) refers to a non-naturally occurring preparation of Ab molecules of single molecular composition, i.e., Ab molecules whose primary sequences are essentially identical, which exhibits a single binding specificity and affinity for a particular epitope. A mAb is an example of an isolated Ab. MAbs may be produced by hybridoma, recombinant, transgenic or other techniques known to those skilled in the art.

A “chimeric” Ab refers to an Ab in which the variable regions are derived from one species and the constant regions are derived from another species, such as an Ab in which the variable regions are derived from a mouse Ab and the constant regions are derived from a human Ab.

A “human” mAb (HuMAb) refers to a mAb having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human” Ab, as used herein, is not intended to include Abs in which CDR sequences derived from the germline of another mammalian species, such as a mouse, have been grafted onto human framework sequences. The terms “human” Abs and “fully human” Abs are used synonymously.

A “humanized” mAb refers to a mAb in which some, most or all of the amino acids outside the CDR domains of a non-human mAb are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

An “anti-antigen” Ab refers to an Ab that binds specifically to an antigen. For example, an anti-PD-1 Ab is an Ab that binds specifically to PD-1, whereas an anti-CXCR4 Ab is an Ab that binds specifically to CXCR4. As used herein, an “anti-PD-1/anti-PD-L1” Ab is an Ab that is used to disrupt the PD-1/PD-L1 signaling pathway, which is an anti-PD-1 Ab or an anti-PD-L1 Ab. Similarly, an “anti-CXCR4/anti-CXCL12” Ab is an Ab that is used to disrupt the CXCR4/CXCL12 signaling pathway, which is an anti-CXCR4 Ab or an anti-CXCL12 Ab.

An “antigen-binding portion” of an Ab (also called an “antigen-binding fragment”) refers to one or more fragments of an Ab that retain the ability to bind specifically to the antigen bound by the whole Ab.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

“C-X-C Chemokine Receptor 4” (CXCR4; also known in the art as, for example, LESTR, Fusin or CD184) refers to a 7-transmembrane G-protein coupled receptor expressed on leukocytes, platelets and other non-hematopoietic cells that comprise the tumor stromal microenvironment. It is also over-expressed in the majority of human cancers and on Tregs and MDSCs. CXCR4 binds to a single ligand, CXCL12. The term “CXCR4” as used herein includes human CXCR4 (hCXCR4), variants, isoforms, and species homologs of hCXCR4, and analogs having at least one common epitope with hCXCR4. The complete hCXCR4 amino acid sequence can be found under GENBANK® Accession No. CAA12166.

“C-X-C motif chemokine 12” (CXCL12; also known as stromal cell-derived factor 1 or SDF-1) is a chemokine that is the only known ligand for the CXCR4 receptor though it may also serve as a ligand for a second receptor, CXCR7 (RDC1). CXCL12 is strongly chemotactic for lymphocytes, and plays an important role in angiogenesis by recruiting endothelial progenitor cells from the bone marrow through a CXCR4-dependent mechanism. It is also thought to be involved in directing metastasis of CXCR4⁺ tumor cells to organs such as lymph node, lung, liver and bone that highly express CXCL12. The term “CXCL12” as used herein includes human CXCL12 (hCXCL12), variants, isoforms, and species homologs of hCXCL12, and analogs having at least one common epitope with hCXCL12. Human CXCL12 is produced in three forms, CXCL12a, CXCL12b and CXCL12c, by alternate splicing of the same gene. The complete amino acid sequence of exemplary CXCL12a, CXCL12b and CXCL12c isoforms can be found under GENBANK® Accession Nos. NP 954637, NP_000600 and NP_001029058, respectively.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, including the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family that is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 amino acid sequence can be found under GENBANK® Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GENBANK® Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms “subject” and “patient” are used interchangeably herein.

A “therapeutically effective amount” or “therapeutically effective dosage” of a drug or therapeutic agent is any amount of the drug or agent that, when used alone or in combination with another therapeutic agent, protects a subject against the onset of a disease or promotes disease regression evidenced by a decrease in severity of disease symptoms, an increase in frequency and duration of disease symptom-free periods, or a prevention or reduction of impairment or disability due to the disease affliction. In addition, the terms “effective” and “effectiveness” with regard to a treatment includes both pharmacological effectiveness and physiological safety. Pharmacological effectiveness refers to the ability of the drug to promote disease regression, e.g., cancer regression, in the patient. Physiological safety refers to an acceptable level of toxicity, or other adverse physiological effects at the cellular, organ and/or organism level (adverse effects) resulting from administration of the drug. The efficacy of a therapeutic agent can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred embodiments of the invention, tumor regression may be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents may require long-term monitoring of the effects of these agents on the target disease.

A therapeutically effective amount of a drug includes a “prophylactically effective amount,” which is any amount of the drug that, when administered alone or in combination with an another therapeutic agent to a subject at risk of developing a disease (e.g., a subject having a pre-malignant condition who is at risk of developing a cancer) or of suffering a recurrence of the disease, inhibits the development or recurrence of the disease (e.g., a cancer). In preferred embodiments, the prophylactically effective amount prevents the development or recurrence of the disease entirely. “Inhibiting” the development or recurrence of a disease means either lessening the likelihood of the disease's development or recurrence, or preventing the development or recurrence of the disease entirely.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “about” refers to a numeric value, composition or characteristic that is within an acceptable error range for the particular value, composition or characteristic as determined by one of ordinary skill in the art, which will depend in part on how the value, composition or characteristic is measured or determined, i.e., the limitations of the measurement system. For example, “about” can mean within 1 or within more than 1 standard deviation per the practice in the art. Alternatively, it can mean a range of plus or minus 20%, more usually a range of plus or minus 10%. When particular values, compositions or characteristics are provided in the application and claims, unless otherwise stated, the meaning of “about” should be assumed to be within an acceptable error range for that particular value, composition or characteristic.

The term “substantially the same” or “essentially the same” refers to a sufficiently high degree of similarity between two or more numeric values, compositions or characteristics that one of skill in the art would consider the difference between these values, compositions or characteristics to be of little or no biological and/or statistical significance within the context of the property being measured. The difference between numeric values being measured may, for example, be less than about 50%, preferably less than about 30%, and more preferably less than about 10%.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Therapeutic Methods

This disclosure provides a method for treating a subject afflicted with a cancer comprising administering to the subject a combination of therapeutically effective amounts of: (a) an Ab or an antigen-binding portion thereof that binds specifically to PD-1 or to PD-L1; and (b) an Ab or an antigen-binding portion thereof that binds specifically to CXCR4 or to CXCL2. In preferred embodiments of any of the present methods, the subject is a human patient.

The present disclosure provides a method for treating a subject afflicted with a cancer comprising administering to the subject a combination of therapeutically effective amounts of: (a) an Ab or an antigen-binding portion thereof that binds specifically to PD-1 or to PD-L1; and (b) an Ab or an antigen-binding portion thereof that binds specifically to CXCR4 or to CXCL2. In certain embodiments, the Ab that binds to PD-1 or to PD-L1 disrupts the interaction between PD-1 and inhibits PD-1/PD-L1 signaling. In other embodiments, the Ab that binds to CXCR4 or CXCL2 disrupts the interaction between CXCR4 and CXCL12 and inhibits CXCR4/CXCL12 signaling.

In certain embodiments of the disclosed methods, the Ab or antigen-binding portion thereof that the Ab that binds to PD-1 or to PD-L1 disrupts the interaction between PD-1 and PD-L1, and thereby inhibits PD-1/PD-L1 signaling.

In certain other embodiments, the Ab or antigen-binding portion thereof that binds to CXCR4 or to CXCL12 disrupts the interaction between CXCR4 and CXCL12, and thereby inhibits CXCR4/CXCL12 signaling. In other embodiments, blockade of the interaction between CXCR4 expressed on immunosuppressant Tregs and/or MDSCs and CXL12 expressed on tumor cells decreases the trafficking of these immunosuppressant cells to the tumor environment, resulting in reduced tumor growth. In yet other embodiments, the Ab that binds specifically to CXCR4 induces apoptosis and/or inhibits growth of CXCR4⁺ tumor cells in vivo (as described in WO 2013/071068). In further embodiments, the anti-CXCR4 Ab comprises an Fc region that mediates effector functions such as Ab-dependent cellular cytotoxicity (ADCC), Ab-dependent cellular phagocytosis (ADCP) and complement-dependent cytotoxicity (CDC) (for example, the Ab is of a human IgG1 or IgG3 isotope), binds to CXCR4 on Tregs and/or MDSCs, and mediates the depletion of these immunosuppressant Tregs and/or MDSCs, thereby enhancing an anti-tumor response). Effector functions mediated by the Fc region can also be increased by certain mutations. Numerous mutations have been made in the CH2 domain of IgG and their effect on ADCC and CDC tested in vitro. For example, an E333A or E333S mutation was reported to increase both ADCC and CDC (Idusogie et al., 2001).

Anti-PD-1 and Anti-PD-L1 Abs Suitable for Use in the Disclosed Methods

Anti-PD-1 Abs suitable for use in the present methods include Abs that bind to PD-1 with high specificity and affinity, block the binding of PD-L1 and/or PD-L2 to PD-1, and inhibit the immunosuppressive effect of the PD-1 signaling pathway. Similarly, anti-PD-L1 Abs suitable for use in these methods are Abs that bind to PD-L1 with high specificity and affinity, block the binding of PD-L1 to PD-1, and inhibit the immunosuppressive effect of the PD-1 signaling pathway. In any of the therapeutic methods disclosed herein, an anti-PD-1 or anti-PD-L1 Ab includes an antigen-binding portion or fragment that binds to the PD-1 receptor or PD-L1 ligand, respectively, and exhibits functional properties similar to those of whole Abs in inhibiting receptor-ligand binding and reversing the inhibition of T cell activity, thereby upregulating an immune response.

Anti-PD-1 Abs

MAbs that bind specifically to PD-1 with high affinity have been disclosed in U.S. Pat. No. 8,008,449. Other anti-PD-1 mAbs have been described in, for example, U.S. Pat. Nos. 7,488,802, 8,168,757 and 8,354,509, and PCT Publication No. WO 2012/145493. The anti-PD-1 mAbs disclosed in U.S. Pat. No. 8,008,449 have been demonstrated to exhibit several or all of the following characteristics: (a) binding to human PD-1 with a K_{D} of about 5×10⁻⁹ M or lower, as determined by the surface plasmon resonance (Biacore) biosensor system; (b) not substantially binding to human CD28, CTLA-4 or ICOS; (c) increasing T-cell proliferation, interferon-γ production and IL-2 secretion in a Mixed Lymphocyte Reaction (MLR) assay; (d) binding to human PD-1 and cynomolgus monkey PD-1; (e) inhibiting the binding of PD-L1 and PD-L2 to PD-1; (f) releasing inhibition imposed by Treg cells on proliferation and interferon-γ production of CD4⁺CD25⁻ T cells; (g) stimulating antigen-specific memory responses; (h) stimulating Ab responses; and (i) inhibiting tumor cell growth in vivo. Anti-PD-1 Abs usable in the disclosed methods of treatment include mAbs that bind specifically to human PD-1 with high affinity and exhibit at least five, and preferably all, of the preceding characteristics. For example, an anti-PD-1 Ab suitable for use in the therapeutic methods disclosed herein (a) binds to human PD-1 with a K_{D} of about 5×10⁻⁹ to 1×10⁻¹⁰ M, as determined by surface plasmon resonance (Biacore); (b) increases T-cell proliferation, interferon-γ production and IL-2 secretion in a MLR assay; (c) inhibits the binding of PD-L1 and PD-L2 to PD-1; (d) reverses inhibition imposed by Tregs on proliferation and interferon-γ production of CD4⁺CD25⁻ T cells; (e) stimulates antigen-specific memory responses; and (f) inhibits tumor cell growth in vivo.

Anti-PD-1 Abs usable in the disclosed methods also include isolated Abs that bind specifically to human PD-1 and cross-compete for binding to human PD-1 with any one of the following anti-PD-1 reference Abs: nivolumab (5C4), the mAbs designated 17D8, 2D3, 4H1, 4A11, 7D3 and 5F4 (see, e.g., U.S. Pat. No. 8,008,449; WO 2013/173223), and pembrolizumab (designated h409A11 in U.S. Pat. No. 8,354,509). The ability of Abs to cross-compete for binding to an antigen, e.g., PD-1, indicates that these Abs bind to the same epitope region of the antigen and sterically hinder the binding of other cross-competing Abs to that particular epitope region. These cross-competing Abs are expected to have functional properties very similar to the properties of the reference Abs by virtue of their binding to substantially the same epitope region of PD-1. Abs that cross-compete with a reference Ab, e.g., nivolumab or pembrolizumab, for binding to an antigen, in this case human PD-1, can be readily identified in standard PD-1 binding assays such as Biacore analysis, ELISA assays or flow cytometry (see, e.g., WO 2013/173223).

Anti-PD-1 Abs usable in the methods of the disclosed invention also include antigen-binding portions of the above Abs. It has been amply demonstrated that the antigen-binding function of an Ab can be performed by fragments of a full-length Ab.

Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab.

These fragments, obtained initially through proteolysis with enzymes such as papain and pepsin, have been subsequently engineered into monovalent and multivalent antigen-binding fragments. For example, although the two domains of the Fv fragment, V_{L} and V_{H}, are coded for by separate genes, they can be joined, using recombinant methods, by a synthetic linker peptide that enables them to be made as a single protein chain in which the V_{L} and V_{H} regions pair to form monovalent molecules known as single chain variable fragments (scFv). Divalent or bivalent scFvs (di-scFvs or bi-scFvs) can be engineered by linking two scFvs in within a single peptide chain known as a tandem scFv which contains two V_{H} and two V_{L} regions. ScFv dimers and higher multimers can also be created using linker peptides of fewer than 10 amino acids that are too short for the two variable regions to fold together, which forces the scFvs to dimerize and produce diabodies or form other multimers. Diabodies have been shown to bind to their cognate antigen with much higher affinity than the corresponding scFvs, having dissociation constants up to 40-fold lower than the K_{D} values for the scFvs. Very short linkers (<3 amino acids) lead to the formation of trivalent triabodies or tetravalent tetrabodies that exhibit even higher affinities for to their antigens than diabodies. Other variants include minibodies, which are scFv-C₃ dimers, and larger scFv-Fc fragments (scFv-CH2-CH3 dimers), and even an isolated CDR may exhibit antigen-binding function. These Ab fragments are engineered using conventional recombinant techniques known to those of skill in the art, and the fragments are screened for utility in the same manner as are intact Abs. All of the above proteolytic and engineered fragments of Abs and related variants (see Hollinger and Hudson, 2005; Olafsen and Wu, 2010, for further details) are intended to be encompassed within the term “antigen-binding portion” of an Ab.

In certain embodiments, the anti-PD-1 Ab or antigen-binding portion thereof comprises a heavy chain constant region which is of a human IgG1, IgG2, IgG3 or IgG4 isotype. In certain preferred embodiments, the anti-PD-1 Ab or antigen-binding portion thereof comprises a heavy chain constant region which is of a human IgG4 isotype. In other embodiments, the anti-PD-1 Ab or antigen-binding portion thereof is of a human IgG1 isotype. In certain other embodiments, the IgG4 heavy chain constant region of the anti-PD-1 Ab or antigen-binding portion thereof contains an S228P mutation (numbered using the Kabat system; Kabat et al., 1991) which replaces a serine residue in the hinge region with the proline residue normally found at the corresponding position in IgG1 isotype Abs. This mutation, which is present in nivolumab, prevents Fab arm exchange with endogenous IgG4 Abs, while retaining the low affinity for activating Fc receptors associated with wild-type IgG4 Abs (Wang et al., 2014). In yet other embodiments, the Ab comprises a light chain constant region which is a human kappa or lambda constant region.

In other embodiments of the present methods, the anti-PD-1 Ab or antigen-binding portion thereof is a mAb or an antigen-binding portion thereof. For administration to human subjects, the anti-PD-1 Ab is preferably a chimeric Ab or, more preferably, a humanized or human Ab. Such chimeric, humanized or human mAbs can be prepared and isolated by methods well known in the art, e.g., as described in U.S. Pat. No. 8,008,449.

In certain preferred embodiments of any of the therapeutic methods described herein comprising administration of an anti-PD-1 Ab, the anti-PD-1 Ab is nivolumab. The V_{H} amino acid sequence of nivolumab is provided herein as SEQ ID NO: 1 and the V_{L} amino acid sequence is provided herein as SEQ ID NO: 2. The amino acid sequences of the heavy and light chains of nivolumab are shown in SEQ ID Nos. 3 and 4, respectively. (The sequence shown for the nivolumab heavy chain does not include the encoded carboxy-terminal lysine residue as this lysine gets cleaved off to varying degrees depending on the host cell and culture conditions, but it essentially completely cleaved off in the Chinese Hamster Ovary (CHO) cell lines used for Ab production. The same applies to the heavy chain sequences disclosed herein for the anti-PD-L1 mAb, BMS-936559, the anti-CXCR4 mAb, ulocuplumab, and the anti-CXCL12 mAb, 2A5.) In other preferred embodiments, the anti-PD-1 Ab is pembrolizumab (h409A11 in U.S. Pat. No. 8,354,509). In other embodiments, the anti-PD-1 Ab is chosen from the human Abs 17D8, 2D3, 4H1, 4A11, 7D3 and 5F4 described in U.S. Pat. No. 8,008,449.

Anti-PD-1 Abs comprising V_{H} and V_{L} regions having amino acid sequences that are highly similar or homologous to the amino acid sequences of nivolumab or any of the above anti-PD-1 Abs and which retain the functional properties of these Abs are also suitable for use in the present methods. For example, suitable Abs include mAbs comprising a V_{H} and V_{L} region each comprising consecutively linked amino acids having a sequence that is at least 80% identical to the amino acid sequence set forth in SEQ ID Nos. 1 and/or 2, respectively. In certain embodiments, the V_{H} and/or V_{L} amino acid sequences exhibits at least 85%, 90%, 95%, or 99% identity to the sequences set forth in SEQ ID Nos. 1 and/or 2, respectively. As used herein, the percent sequence identity between two amino acid sequences is a function of the number of identical positions shared by the sequences relative to the length of the sequences compared (i.e., % identity=number of identical positions/total number of positions being compared×100), taking into account the number of any gaps, and the length of each such gap, introduced to maximize the degree of sequence identity between the two sequences. The comparison of sequences and determination of percent identity between two sequences can be accomplished using mathematical algorithms that are well known to those of ordinary skill in the art (see, e.g., U.S. Pat. No. 8,008,449).

Anti-PD-L1 Abs

Because anti-PD-1 and anti-PD-L1 target the same signaling pathway and have been shown in clinical trials to exhibit comparable levels of efficacy in a variety of cancers (see, e.g., Brahmer et al., 2012; Topalian et al., 2012b; WO 2013/173223), an anti-PD-L1 Ab may be substituted for the anti-PD-1 Ab in the combination therapy methods disclosed herein.

MAbs that bind specifically to PD-L1 with high affinity have been disclosed in U.S. Pat. No. 7,943,743. Other anti-PD-L1 mAbs have been described in, for example, U.S. Pat. No. 8,217,149 and PCT Publication Nos. WO 2011/066389, WO 2012/145493, WO 2013/079174 and WO 2013/181634. The anti-PD-1 HuMAbs disclosed in U.S. Pat. No. 7,943,743 have been demonstrated to exhibit one or more of the following characteristics: (a) binding to human PD-L1 with a K_{D} of about 5×10⁻⁹ M or lower, as determined by surface plasmon resonance; (b) increasing T-cell proliferation, interferon-γ production and IL-2 secretion in a MLR assay; (c) stimulating Ab responses; (d) inhibiting the binding of PD-L1 to PD-1; and (e) reversing the suppressive effect of Tregs on T cell effector cells and/or dendritic cells. Anti-PD-L1 Abs for use in the therapeutic methods disclosed herein include Abs that bind specifically to human PD-L1 with high affinity and exhibit at least three, and preferably all, of the preceding characteristics. For example, an anti-PD-L1 Ab suitable for use in these methods (a) binds to human PD-1 with a K_{D} of about 5×10⁻⁹ to 1×10⁻¹⁰ M, as determined by surface plasmon resonance (Biacore); (b) increases T-cell proliferation, interferon-γ production and IL-2 secretion in a MLR assay; (c) inhibits the binding of PD-L1 to PD-1; and (d) reverses the suppressive effect of Tregs on T cell effector cells and/or dendritic cells.

A preferred anti-PD-L1 Ab for use in the present methods is BMS-936559 (formerly MDX-1105; designated 12A4 in U.S. Pat. No. 7,943,743). The V_{H} and V_{L} amino acid sequences of BMS-936559 are set forth in SEQ ID Nos. 5 and 6, respectively, and sequences of the heavy and light chains of BMS-936559 are shown in SEQ ID Nos. 7 and 8, respectively. Other anti-PD-L1 Abs suitable for use in the present methods include mAbs comprising a V_{H} and V_{L} region each having an amino acid sequence that is at least 80% identical to the amino acid sequence set forth in SEQ ID Nos. 5 and/or 6, respectively, and which retain the functional properties of BMS-936559. In certain embodiments, the V_{H} and/or V_{L} amino acid sequences exhibit at least 85%, 90%, 95%, or 99% identity to the sequences set forth in SEQ ID Nos. 5 and/or 6, respectively. Yet other suitable anti-PD-L1 Abs include atezolizumab (formerly MPDL3280A; Herbst et al., 2014; designated YW243.55 S70 in U.S. Pat. No. 8,217,149), durvalumab (formerly MEDI4736; Segal et al., 2014; designated 2.14H9OPT in WO 2011/066389), STI-A1014 (designated H6 in WO 2013/181634), and avelumab (designated A09-246-2 in WO 2013/079174).

Anti-PD-L1 Abs suitable for use in the disclosed methods also include isolated Abs that bind specifically to human PD-L1 and cross-compete for binding to human PD-L1 with any one of the following reference Abs: BMS-936559 (12A4), the Abs designated 3G10, 10A5, 5F8, 10H10, 1B12, 7H1, 11E6, 12B7 and 13G4 (see, e.g., U.S. Pat. No. 7,943,743; WO 2013/173223), atezolizumab (YW243.55S70 in U.S. Pat. No. 8,217,149), durvalumab (2.14H9OPT in WO 2011/066389), STI-A1014 (H6 in WO 2013/181634), and avelumab (A09-246-2 in WO 2013/079174). The ability of an Ab to cross-compete with a reference Ab for binding to human PD-L1 demonstrates that such Ab binds to the same epitope region of PD-L1 as the reference Ab and is expected to have very similar functional properties to that of the reference Ab by virtue of its binding to substantially the same epitope region of PD-L1. For example, cross-competing anti-PD-L1 mAbs 3G10, 1B12, 13G4, 12A4 (BMS-936559), 10A5, 12B7, 11E6 and 5F8 (see WO 2013/173223) have been shown to have similar functional properties (see U.S. Pat. No. 7,943,743 at Examples 3-11), whereas mAb 10H10, which binds to a different epitope region (see WO 2013/173223), behaves differently (U.S. Pat. No. 7,943,743 at Example 11). Cross-competing Abs can be identified in standard PD-L1 binding assays that are well known to persons skilled in the art.

In certain preferred embodiments, the anti-PD-L1 Abs for use in the present methods are mAbs. In other preferred embodiments, these cross-competing Abs are chimeric Abs, humanized or human Abs. Chimeric, humanized and human Abs can be prepared and isolated by methods well known in the art, e.g., as described in U.S. Pat. No. 7,943,743.

In certain embodiments, the anti-PD-L1 Ab or antigen-binding portion thereof comprises a heavy chain constant region which is of a human IgG1, IgG2, IgG3 or IgG4 isotype. In certain other embodiments, the anti-PD-L1 Ab or antigen-binding portion thereof is of a human IgG1 of IgG4 isotype. In further embodiments, the sequence of the IgG4 heavy chain constant region of the anti-PD-L1 Ab or antigen-binding portion thereof contains an S228P mutation. In other embodiments, the Ab comprises a light chain constant region which is a human kappa or lambda constant region.

Anti-PD-L1 Abs of the invention also include antigen-binding portions of the above Abs, including Fab, F(ab′)₂, Fd, Fv, and scFv, di-scFv or bi-scFv, and scFv-Fc fragments, diabodies, triabodies, tetrabodies, and isolated CDRs.

Anti-CXCR4 and Anti-CXCL12 Abs Suitable for Use in the Disclosed Methods

Anti-CXCR4 and anti-CXCL12 Abs suitable for use in the disclosed methods are Abs that bind specifically to CXCR4 and CXCL12, respectively, with high specificity and affinity. In certain embodiments, such anti-CXCR4 Abs block the binding of CXCR4 and CXCL12, and inhibit the activity of CXCR4. In certain other embodiments, the anti-CXCR4 Ab induces apoptosis and/or inhibits growth of CXCR4⁺ tumor cells in vivo. In yet other embodiments, the anti-CXCR4 Ab binds to CXCR4 on Tregs and/or MDSCs and mediates the destruction of these immunosuppressant cells by either direct apoptosis or depletion via ADCC, ADCP and/or CDC mechanisms.

Anti-CXCL12 Abs usable in these methods bind to the CXCL12 ligand with high specificity and affinity. Similar to anti-CXCR4, such anti-CXCL12 Abs block the binding of CXCR4 and CXCL12, and inhibit the activity of the CXCR4 receptor.

Anti-CXCR4 Abs

Anti-CXCR4 mAbs that bind specifically to CXCR4 with high affinity, specifically mAbs F7 (ulocuplumab; also previously designated BMS-936564 and MDX-1338), F9, D1 and E2, have been exemplified in WO 2008/060367. Methods of using these Abs to treat hematological malignancies are also described in WO 2008/060367 and WO 2013/071068. Other anti-CXCR4 mAbs have been described in, for example, WO 2008/142303, WO 2010/037831, WO 2009/140124, WO 2013/013025, and U.S. Publication No. 2015/0037328.

The anti-CXCR4 mAbs disclosed in WO 2008/060367 have been demonstrated to exhibit one or more of the following characteristics: (a) binding to human CXCR4 on a surface of a cell with an EC₅₀ of less than about 100 nM (e.g., about 20-80 nM); (b) inhibiting binding of CXCL12 to CXCR4 with an EC₅₀ of less than about 30 nM (e.g., about 2-29 nM); (c) inhibiting CXCL12-induced calcium flux in cells expressing CXCR4 with an EC₅₀ of less than about 1 nM (e.g., about 0.3-0.9 nM); (d) inhibiting CXCL12-induced migration of cells expressing CXCR4 with an EC₅₀ of less than about 20 nM (e.g., about 12-19 nM); (e) inhibiting capillary tube formation by human umbilical vein endothelial cells; (f) inducing apoptosis in cells expressing CXCR4; (g) inhibiting proliferation of CXCR4⁺ tumor cells in vitro; (h) inhibiting CXCR4⁺ tumor cell proliferation and/or inducing CXCR4⁺ tumor cell apoptosis in vivo; (i) inhibiting metastases of CXCR4⁺ tumor cells; and (j) increasing survival time of a CXCR4⁺ tumor-bearing subject. Anti-CXCR4 Abs usable in the methods of present invention include mAbs that bind specifically to human CXCR4 expressed on a cell surface with high affinity, for example, with a K_{D} of 1×10⁻⁸ M or less, preferably with a K_{D} of 5×10⁻⁹ M or less, and exhibit at least five, and preferably all, of the other preceding characteristics.

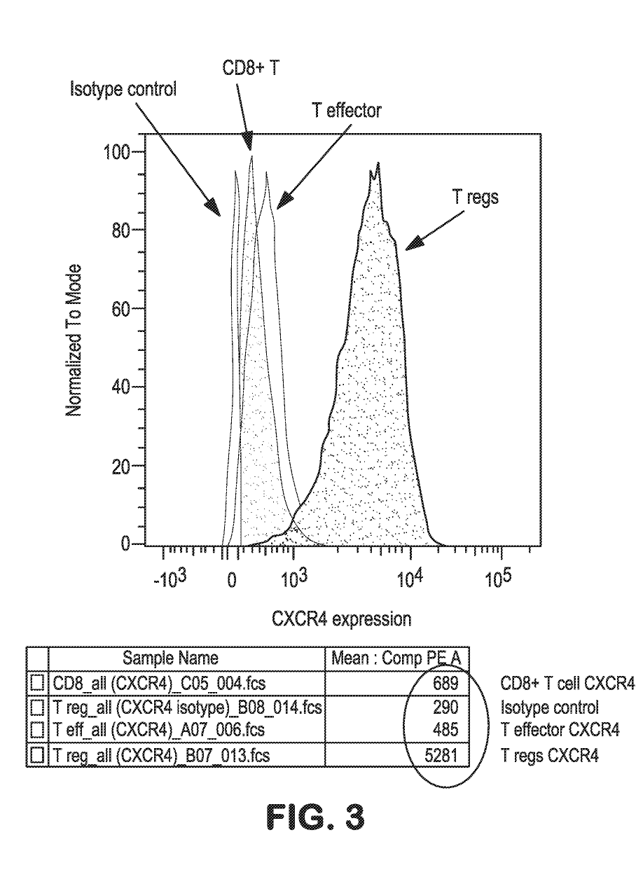

For example, an anti-CXCR4 Ab suitable for use in the disclosed methods of treatment (a) binds to human PD-1 with a K_{D} of about 5×10⁻⁹ to 1×10⁻¹⁰ M, as determined by surface plasmon resonance (Biacore); (b) inhibits binding of CXCL12 to CXCR4 with an EC₅₀ of less than about 10 nM (e.g., about 1-10 nM); (c) induces apoptosis in cells expressing CXCR4; (d) inhibits proliferation of CXCR4⁺ tumor cells in vitro; (e) inhibits CXCR4⁺ tumor cell proliferation and/or induces CXCR4⁺ tumor cell apoptosis in vivo; and (f) inhibits metastases of CXCR4⁺ tumor cells. In certain preferred embodiments, the anti-CXCR4 Ab comprises an Fc region (e.g., human IgG1 or IgG3) that possesses effector functions including ADCC, ADCP and/or CDC and mediates the depletion of immunosuppressant Tregs and/or MDSCs. These immunosuppressant cells are known to overexpress CXCR4 (see FIG. 3). Thus, preferred anti-CXCR4 reverse inhibition imposed by Tregs and/or MDSCs on proliferation and interferon-γ production of CD4⁺CD25⁻ T cells.

A suitable anti-CXCR4 Ab for use in the methods disclosed herein is ulocuplumab, which comprises V_{H} and V_{L} regions having the amino acid sequences set forth in SEQ ID Nos. 9 and 10, respectively, corresponding to the V_{H} and V_{L} sequences of F7GL in WO 2008/060367. (As described in WO 2008/060367, the N-terminal (FR1) region of the V_{H} and V_{L} regions of the exemplified anti-CXCR4 Abs, F7, F9, D1 and E2, contained amino acid substitutions compared to the germline sequences from which they were derived because these non-germline residues were encoded by the primers used to create the phage display libraries from which genes encoding the Abs were isolated. The substituted framework residues in the N-terminal regions of the V_{H} and V_{L} regions were “back-mutated” to restore the f germline sequences (referred to as “GL” forms, for germline), and these “back-mutated” sequences are present in ulocuplumab. The sequences disclosed herein for the 2A5 heavy and light chains similarly reflect the “back-mutation” of the N-terminal FR1 regions to their germline configuration; see U.S. Pat. No. 8,496,931). The sequences of the complete heavy and light chains of ulocuplumab are set forth in SEQ ID Nos. 11 and 12, respectively. Other suitable anti-CXCR4 Abs include, for example, the Abs designated c414H5 and c515H7 (WO 2010/037831), the Abs designated Antibody I, Antibody II, Antibody III, Antibody IV, and Antibody V (U.S. Pat. No. 7,892,546), the Ab designated 6C7 (WO 2013/013025), and humanized 3G10 Abs, e.g., the Abs designated h3G1 0.A57.A58, h3G10.1.91.A58A and h3G10.1.91.A58B (U.S. Publication No. 2015/0037328).

Related anti-CXCR4 Abs comprising V_{H} and V_{L} regions having amino acid sequences that are at least 80% identical to the amino acid sequence set forth in SEQ ID Nos. 11 and/or 12, respectively, and which retain the functional properties of ulocuplumab are also suitable for use in the present methods. In certain embodiments, the V_{H} and/or V_{L} amino acid sequences exhibit at least 85%, 90%, 95%, or 99% identity to the sequences set forth in SEQ ID Nos. 11 and/or 12, respectively.

The data from mouse tumor models disclosed herein indicate that an anti-CXCR4 Ab comprising an Fc region that mediates effector functions is able to synergize with an anti-PD-1 Ab to produce a significantly enhanced anti-tumor effect (see Examples 2-5). Accordingly, in certain preferred embodiments, the anti-CXCR4 Ab suitable for use in the disclosed methods comprises an Fc region (e.g., human IgG1 or IgG3) that possesses effector functions. For example, the heavy chain sequence of the human IgG1f variant of ulocuplumab is set forth in SEQ ID NO: 13, and the heavy chain sequence of the human IgG3b0 variant of ulocuplumab is set forth in SEQ ID NO: 14. The corresponding light chain sequences of these IgG1 and IgG3 variants would be the same as in ulocuplumab, i.e., the sequence set forth in SEQ ID NO: 12.

Additional anti-CXCR4 Abs usable in the disclosed methods include Abs that bind specifically to human CXCR4 and cross-compete for binding to human CXCR4 with a reference Ab which is ulocuplumab (F7) or any of the Abs designated F9, D1 and E2 (see, e.g., WO 2008/060367; WO 2013/071068). These cross-competing Abs are expected to have functional properties very similar those of ulocuplumab, F9, D1 or E2, respectively, by virtue of their binding to substantially the same epitope region of CXCR4. Cross-competing Abs can be readily identified based on their ability to cross-compete with a reference Ab, e.g., ulocuplumab, in standard CXCR4 binding assays such as Biacore analysis, ELISA assays or flow cytometry.

The anti-CXCR4 Abs suitable for use in the disclosed methods are preferably mAbs. In certain embodiments, the anti-CXCR4 Ab or antigen-binding portion thereof is a chimeric, humanized or human monoclonal Ab or a portion thereof. In certain preferred embodiments for treating a human subject, the Ab is a humanized Ab. In other preferred embodiments, the Ab is a human Ab. Such chimeric, humanized or human mAbs can be prepared and isolated by methods well known in the art, e.g., as described in WO 2008/060367.

In certain embodiments, the anti-CXCR4 Ab or antigen-binding portion thereof is of a human IgG1, IgG2, IgG3 or IgG4 isotype. In further embodiments, the Ab or antigen-binding portion thereof is of a human IgG1 of IgG4 isotype. In certain embodiments, the IgG4 heavy chain constant region of the anti-CXCR4 Ab or antigen-binding portion thereof contains an S228P mutation. In certain preferred embodiments, the Ab or antigen-binding portion thereof comprises an Fc region that mediates effector functions, for example it is of a human IgG1 or human IgG3 isotype, or comprises a mutation (e.g., E333A or E333S; Idusogie et al., 2001) that increases effector functions. In other embodiments, the Ab comprises a light chain constant region which is a human kappa or lambda constant region.

Anti-CXCR4 Abs usable in the methods of the disclosed invention also include antigen-binding portions of the above Abs, such as Fab, F(ab′)₂, Fd, Fv, and scFv, di-scFv or bi-scFv, and scFv-Fc fragments, diabodies, triabodies, tetrabodies, and isolated CDRs.

Anti-CXCL12 Abs

MAbs that bind specifically to CXCL12 with high affinity have been disclosed in U.S. Pat. No. 8,496,931. These anti-CXCL12 mAbs disclosed in U.S. Pat. No. 8,496,931 have been demonstrated to exhibit one or more of the following characteristics: (a) binding to human CXCL12 with a K_{D} of about 1.3×10⁻⁹ M or lower, as determined by surface plasmon resonance; (b) blocking the binding of CXCL12 to CEM (human T cell leukemia) cells; (c) blocking CXCL12-induced calcium flux in CEM cells; (d) blocking CXCL12-induced migration of CEM cells; and (e) blocking capillary tube formation in HuVEC cells. This indicates that anti-CXCL12 exhibits several of the properties of anti-CXCR4 such as blocking the binding of CXCL12 to CXCR4, blocking CXCL12-induced calcium flux in, and blocking CXCL12-induced migration of, CXCR4-expressing cells. However, unlike anti-CXCR4, anti-CXCL12 was shown to not inhibit tumor growth cells, leading to the conclusion that anti-tumor control is not dependent on blockade of the CXCL12/CXCR4 axis (WO 2013/071068). In contrast, Pitt et al. (2015) reported that Cxcl12 deletion from vascular endothelial cells impeded growth of T cell acute lymphoblastic leukemia (T-ALL) tumor cells. In any event, as discussed herein, the rationale for combining blockade of the PD-1 and CXCR4 signaling pathways is not dependent on anti-tumor activity of the CXCR4 blocker, but may rely more on the ability of the CXCR4/CXCL12 inhibitor to enhance penetration of activated immune cells to the tumor site (see, also, Feig et al., 2013; Fearon, 2014; WO 2015/019284; Chen et al., 2015). Without being bound by any particular theory or mechanism of action, anti-CXCL12 Abs usable in the present invention include mAbs that bind specifically to human CXCL12 and exhibit at least three, and preferably all, of the characteristics of anti-CXCL12 mAbs listed above. A preferred anti-CXCL12 Ab for use in the methods disclosed herein is the mAb designated 2A5 in U.S. Pat. No. 8,496,931. MAb 2A5 comprises a V_{H} and V_{L} region comprising consecutively linked amino acid having the sequences set forth in SEQ ID Nos. 15 and 16, respectively (corresponding to the 2A5 V_{H} and V_{L} sequences in FR1 “back-mutated” to their germline configuration; see U.S. Pat. No. 8,496,931). The sequences of the complete heavy and light chains of mAb 2A5 are set forth in SEQ ID Nos. 17 and 18, respectively. Other usable Abs include the mAbs designated 1D3, 1H2 and 1C6 in U.S. Pat. No. 8,496,931.

Anti-CXCL12 Abs comprising V_{H} and V_{L} regions having amino acid sequences that are at least 80% identical to the amino acid sequence set forth in SEQ ID Nos. 15 and/or 16, respectively, and which retain the functional properties of the 2A5 mAb, are also suitable for use in the present methods. In certain embodiments, the V_{H} and/or V_{L} amino acid sequences exhibit at least 85%, 90%, 95%, or 99% identity to the sequences set forth in SEQ ID Nos. 15 and/or 16, respectively.

Additional anti-CXCL12 Abs suitable for use in the disclosed methods include Abs that bind to substantially the same epitope region of either the monomer or dimer of CXCL12a as mAbs 2A5 and 1C6 on the one hand, or mAbs 1D3 and 1 H2 on the other hand. MAbs 1C6 and 2A5 are recognize two epitope peptides, one near the N-terminal region amino acid residues 7-19, which is also the known receptor binding site, and the other one on the third beta strand between residues 37-50, whereas mAbs 1D3 and 1H2 block the heparin binding site, and appear to bind predominantly to the CXCL12a dimer interface binding site, between residues 24-30 of the first and the second monomer where heparin also binds (U.S. Pat. No. 8,496,931). The Arg8 residue is critical in epitope binding by mAbs 1C6 and 2A5. Abs that bind to the same epitope region of CXCL12 are expected to have functional properties very similar those of the 1C6/2A5 and 1D3/12 reference Abs, respectively.

Also suitable for use in the disclosed methods are Abs that bind specifically to human CXCL12 and cross-compete for binding to human CXCL12 with any of the Abs designated 1D3, 1H2, 1C6 and 2A5 (see U.S. Pat. No. 8,496,931). These cross-competing Abs are expected to have functional properties very similar those of 1D3, 1H2, 1C6 and 2A5, respectively, by virtue of their binding to substantially the same epitope region of CXCL12. Such cross-competing anti-CXCL12 Abs can be readily identified based on their ability to cross-compete with 1D3, 1H2, 1C6 or 2A5 in standard CXCL12 binding assays such as Biacore analysis, ELISA assays or flow cytometry (see U.S. Pat. No. 8,496,931).

In preferred embodiments, the anti-CXCL12 Abs suitable for use in the disclosed methods are mAbs. In certain embodiments, these anti-CXCL12 Abs are chimeric Abs, preferably humanized Abs, or more preferably human Abs. Such chimeric, humanized or human mAbs can be prepared and isolated by methods well known in the art, e.g., as described in U.S. Pat. No. 8,496,931.

In certain embodiments, the anti-CXCL12 Ab or antigen-binding portion thereof comprises a heavy chain constant region which is of a human IgG1, IgG2, IgG3 or IgG4 isotype. In certain other embodiments, the anti-CXCL12 Ab or antigen-binding portion thereof is of a human IgG1 of IgG4 isotype. In further embodiments, the sequence of the IgG4 heavy chain constant region of the anti-CXCL12 Ab or antigen-binding portion thereof contains an S228P mutation. In yet other embodiments, the Ab comprises a light chain constant region which is a human kappa or lambda constant region.

Antigen-binding portions of the above anti-CXCL12 Abs may also be used, such as Fab, F(ab′)₂, Fd, Fv, and scFv, di-scFv or bi-scFv, and scFv-Fc fragments, diabodies, triabodies, tetrabodies, and isolated CDRs.

Cross-Competing Abs

The ability of a pair of Abs to “cross-compete” for binding to an antigen indicates that a first Ab binds to substantially the same epitope region of the antigen as, and reduces the binding of, a second Ab to that particular epitope region and, conversely, the second Ab binds to substantially the same epitope region of the antigen as, and reduces the binding of, the first Ab to that epitope region. Thus, the ability of a test Ab to competitively inhibit the binding of, for example, nivolumab to human PD-1, demonstrates that the test Ab binds to substantially the same epitope region of human PD-1 as does nivolumab.

A first Ab is considered to bind to “substantially the same epitope” or “substantially the same determinant” as does a second Ab if the first Ab reduces the binding of the second Ab to an antigen by at least about 40%. Preferably, the first Ab reduces the binding of the second Ab to the antigen by more than about 50% (e.g., at least about 60% or at least about 70%). In more preferred embodiments, the first Ab reduces the binding of the second Ab to the antigen by more than about 70% (e.g., at least about 80%, at least about 90%, or about 100%). The order of the first and second Abs can be reversed, i.e. the “second” Ab can be first bound to the surface and the “first” is thereafter brought into contact with the surface in the presence of the “second” Ab. The Abs are considered to “cross-compete” if a competitive reduction in binding to the antigen is observed irrespective of the order in which the Abs are added to the immobilized antigen.

Cross-competing Abs are expected to have similar functional properties by virtue of their binding to substantially the same epitope region of an antigen such as a PD-1 or CXCR4 receptor. The higher the degree of cross-competition, the more similar will the functional properties be. For example, two cross-competing Abs are expected to have essentially the same functional properties if they each inhibit binding of the other to an epitope by at least about 80%. This similarity in function is expected to be even closer if the cross-competing Abs exhibit similar affinities for binding to the epitope as measured by the dissociation constant (K_{D}).

Cross-competing anti-antigen Abs can be readily identified based on their ability to detectably compete in standard antigen binding assays, including surface plasmon resonance (BIAcore®) analysis, ELISA assays or flow cytometry, using either recombinant antigen molecules or cell-surface expressed antigen molecules. By way of example, a simple competition assay to identify whether a test Ab competes with nivolumab for binding to human PD-1 may involve: (1) measuring the binding of nivolumab, applied at saturating concentration, to a BIAcore chip (or other suitable medium for surface plasmon resonance analysis) onto which human PD-1 is immobilized, and (2) measuring the binding of nivolumab to a human PD-1-coated BIAcore chip (or other medium suitable) to which the test Ab has been previously bound. The binding of nivolumab to the PD-1-coated surface in the presence and absence of the test Ab is compared. A significant (e.g., more than about 40%) reduction in binding of nivolumab in the presence of the test Ab indicates that both Abs recognize substantially the same epitope such that they compete for binding to the KIR2DL1 target. The percentage by which the binding of a first Ab to an antigen is inhibited by a second Ab can be calculated as: [1-(detected binding of first Ab in presence of second Ab)/(detected binding of first Ab in absence of second Ab)]×100. To determine whether the Abs cross-compete, the competitive binding assay is repeated except that the binding of the test Ab to the PD-1-coated chip in the presence of nivolumab is measured.

Cancers Amenable to Treatment by Disclosed Methods Immuno-oncology, which relies on using the practically infinite flexibility of the immune system to attack and destroy cancer cells, is applicable to treating a very broad range of cancers (see, e.g., Callahan et al., 2016; Vick and Mahadevan, 2016; Lesokhin et al., 2015; Yao et al., 2013; Chen and Mellman, 2013; Pardoll, 2012). The anti-PD-1 Ab, nivolumab, has been shown to be effective in inhibiting many different types of cancers (see, e.g., Topalian et al., 2012b; WO 2013/173223), and is currently undergoing clinical trials in multiple solid and hematological cancers. Accordingly, the disclosed methods employing dual blockade of the PD-1/PD-L1 and CXCR4/CXCL12 signaling pathways are applicable to treating a wide variety of both solid and liquid tumors. The initial focus of these methods, however, is for the treatment of two solid tumors, SCLC and PAC, for which there is a large unmet need for effective therapies.

Unmet medical need in small cell lung cancer (SCLC) Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES@, 2015). SCLC accounts for approximately 15% of new cases of lung cancer, and an estimated 31,000 cases are expected to be diagnosed in the United States in 2015 (Siegel et al., 2015; NCCN GUIDELINES@, Version 1.2016—Small Cell Lung Cancer). When compared with NSCLC, SCLC generally has a more rapid doubling time, a higher growth fraction, and earlier development of widespread metastases. In patients with limited stage (LD) disease, the goal of treatment is cure using chemotherapy plus thoracic radiotherapy (NCCN GUIDELINES@, Version 1.2016—Small Cell Lung Cancer; Sorensen et al., 2010). In patients with extensive stage (ED) disease, chemotherapy can prolong survival in most patients; however, long term survival is rare (NCCN GUIDELINES@, Version 1.2016—Small Cell Lung Cancer; Sorensen et al., 2010; Janne et al., 2002; Chute et al., 1999). Despite the activity of several agents in SCLC, an etoposide and platinum (e.g., cisplatin)-containing regimen remains standard for SCLC because of its higher activity compared to other chemotherapy regimens and the ease of combining it with radiation. Initial response rates can be robust with 70-90% responders in LD-SCLC and 50-70% responders in ED-SCLC (Califano et al., 2012). However, disease typically recurs rapidly which is reflected by the median survival rates of 9 to 11 months for ED-SCLC and the 2-year survival rate is less than 5% (NCCN GUIDELINES@, Version 1.2016—Small Cell Lung Cancer; Sorensen et al., 2010). Second-line (2L) therapy generally involves single-agent chemotherapy and provides palliative care in many patients. Innovative treatment strategies that can enhance the clinical benefit and prolong survival and quality of life in SCLC are urgently needed.

Rationale for Combined Blockade of PD-1 and CXCR4 Signaling in SCLC

Nivolumab and pembrolizumab have been approved for treatment of NSCLC, and several checkpoint inhibitors are being evaluated in both NSCLC and SCLC. The preliminary efficacy observed has supported further evaluation in both forms of lung cancer. A randomized Phase 2 trial in SCLC demonstrated that ipilimumab (10 mg/kg), in combination with paclitaxel/carboplatin, significantly prolonged progression-free survival (PFS) in the front-line setting (Reck et al., 2013). A Phase 3 study is ongoing comparing ipilimumab in combination with etoposide/carboplatin or etoposide/cisplatin as first-line (1L) treatment in ED-SCLC (NCT01450761). Similarly, nivolumab has been approved in squamous and non-squamous NSCLC, and early trials are being evaluated in SCLC patients that have failed prior chemotherapy (Topalian et al., 2012b; NCT01928394). While the efficacy of checkpoint inhibitors in these trials is promising, the combination with other novel targeted agents may be required to maximize response rates and/or improve survival outcomes.

In SCLC, the tumor stroma contributes to the refractory nature of SCLC and therapies that target the stromal compartment are being evaluated in this disease (Burger and Kipps, 2006; Burger et al., 2011). CXCR4 is a stromal cell marker that is overexpressed in a high percentage of primary tumors and cell lines, and constitutive secretion of its ligand, CXCL12, by stromal cells induces migration and adhesion of SCLC cells via CXCR4-dependent pathways (Burger et al., 2003; Gangadhar et al., 2010). Furthermore, stromal cells may protect SCLC from chemotherapy-induced apoptosis which can be antagonized by CXCR4 inhibitors (Hartmann et al., 2005). In a pre-clinical mouse model, a small peptidic CXCR4 inhibitor suppressed pulmonary metastases of CXCR4-expressing SCLC in size and number (Otani et al., 2012), supporting CXCR4 blockade in the treatment of SCLC. In contrast, however, a small molecule CXCR4 inhibitor did not demonstrate efficacy in a recent Phase 2 clinical trial in ED-SCLC patients when combined with chemotherapy (Spigal et al., 2014). Together, these data suggest that additional immune-mediated mechanisms combined with CXCR4-targeting agents may be required to overcome resistance and provide clinical benefit for SCLC patients.

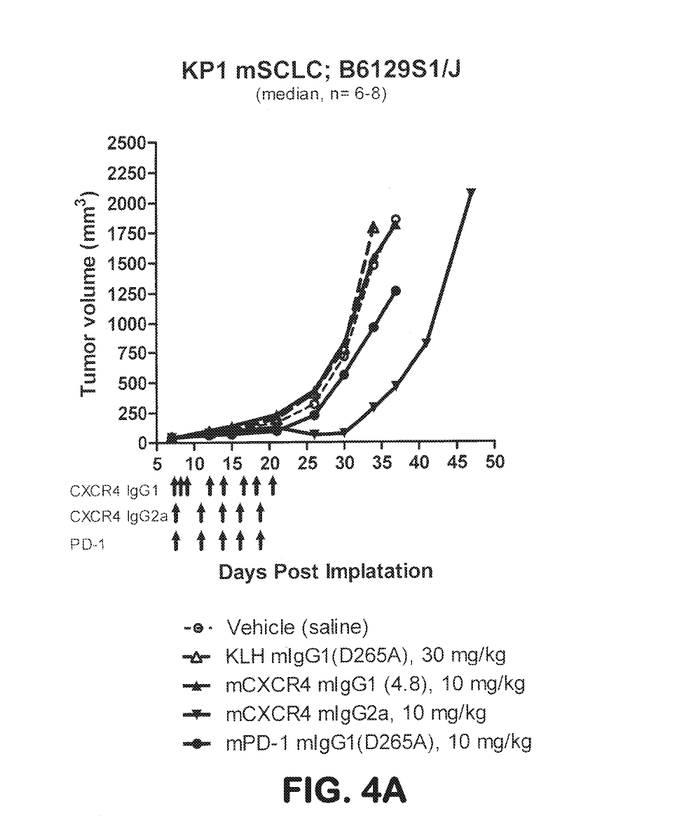

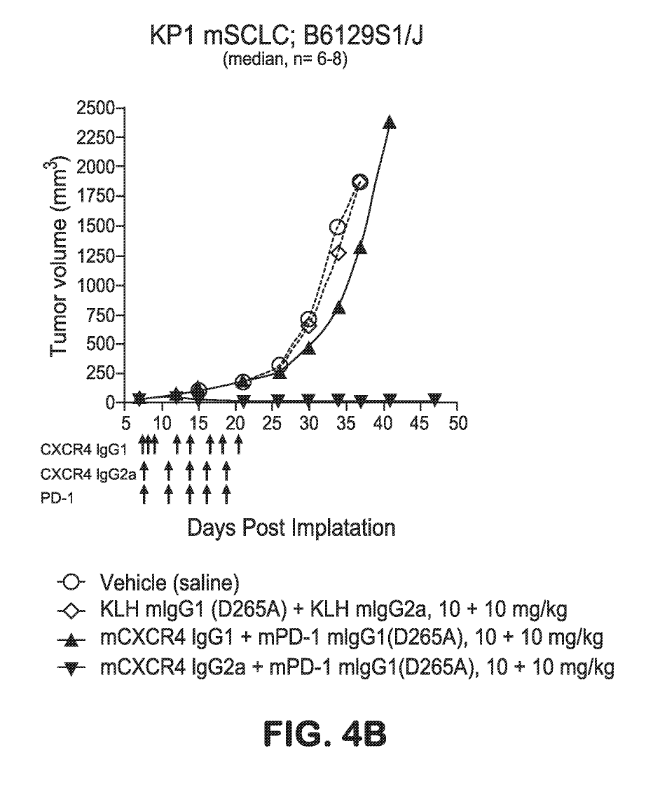

The results of experiments to evaluate the combination of anti-PD-1 and anti-CXCR4 in mouse SCLC, colon and liver cancer models described herein (Examples 2-5) support the efficacy of this combination for treating SCLC. These experiments indicate that anti-PD-1 and anti-CXCR4 interact synergistically to produce anti-tumor effects that are more potent than either antibody alone. The most pronounced synergism was observed with the combination of a depleting mIgG2a anti-CXCR4 Ab in combination with anti-PD-1 in a CXCR4-expressing syngeneic Kp1 tumor model (FIG. 4B). Multiple mechanisms of action may contribute to this strong synergistic interaction. For example, anti-CXCR4 may directly induce apoptosis of tumor cells as shown in WO 2013/071068. Anti-CXCR4-mIgG2a may also mediate the depletion of tumor cells by ADCC, ADCP and/or CDC. The much weaker anti-tumor effect seen with a non-depleting anti-CXCR4-mIgG1 Ab in combination with anti-PD-1 (FIG. 4B) suggests that apoptosis of SCLC cells may not be a major factor in this model system.

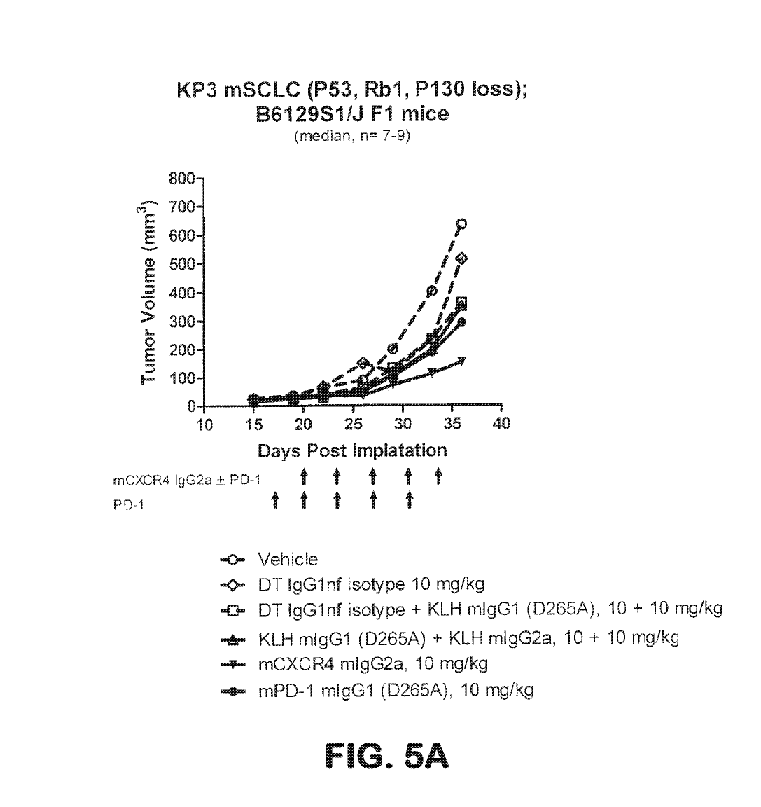

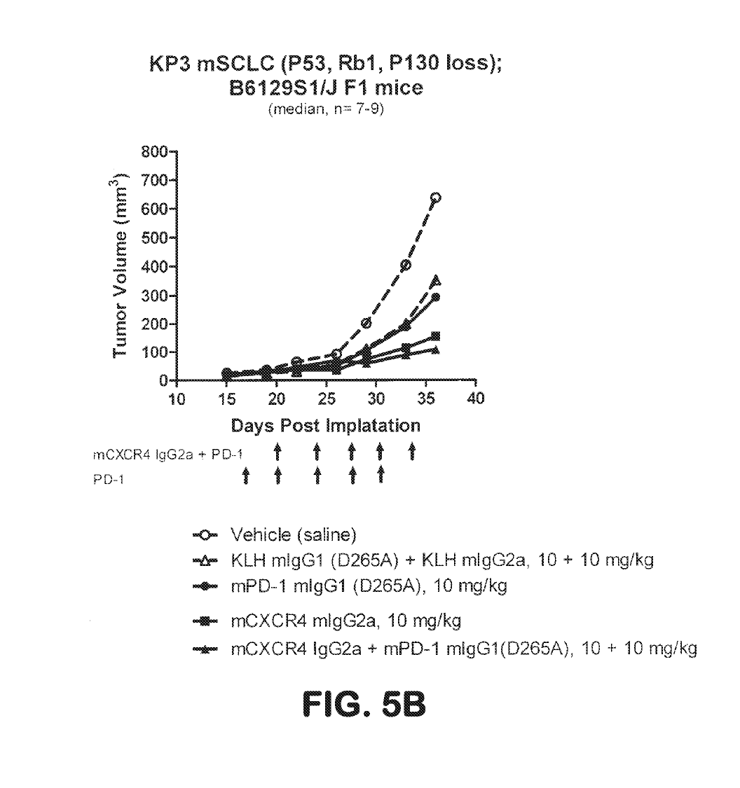

A lower level of synergism was observed in the Kp3 CXCR4-nonexpressing SCLC mouse model (FIG. 5B). But the finding that anti-CXCR4-mIgG2a shows activity as monotherapy (FIG. 5A) and in combination with anti-PD-1 (FIG. 5B) suggests that anti-CXCR4 may act on CXCR4-expressing cells other than the tumors cells themselves. As it is known that the immunosuppressant Tregs (FIG. 3; Wang et al., 2012; Obermajer et al., 2011; Katoh and Watanabe, 2015) and MDSCs express high levels on CXCR4, anti-CXCR4-mediated depletion of Tregs and/or MDSCs may reverse immunosuppression by these cells types and contribute to an anti-tumor effect. Additionally, there is some evidence that Tregs and MDSCs may blunting T cell function via a mechanism involving the PD-1/PD-L1 signaling pathway. Depletion of Tregs and/or MDSCs with a depleting anti-CXCR4 antibody such as anti-CXCR4 IgG2a may indirectly contribute to alleviating the immunoinhibitory effect of PD-1/PD-L1 and thereby potentiate the effects of an anti-PD-1 or anti-PD-L1 Ab.

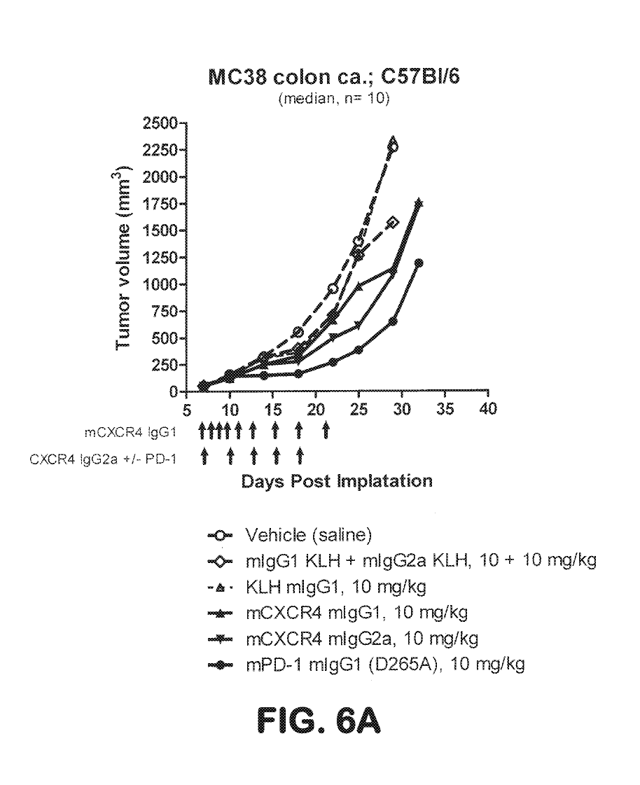

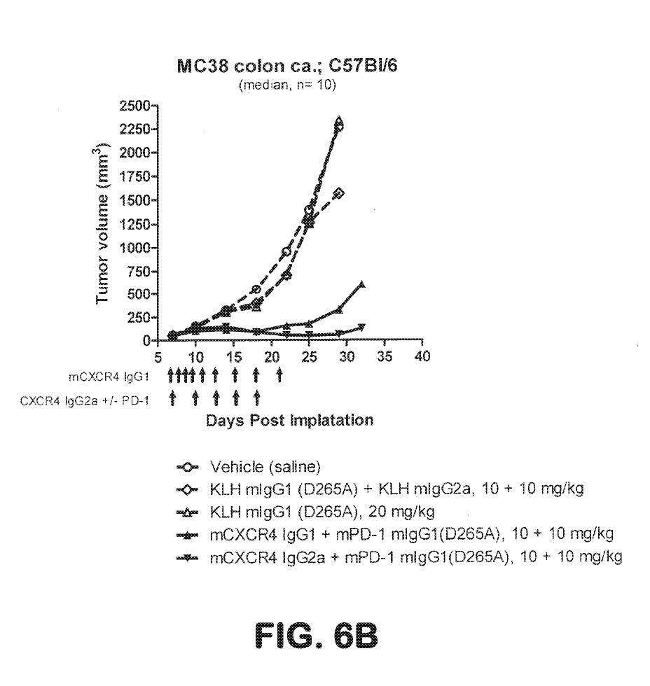

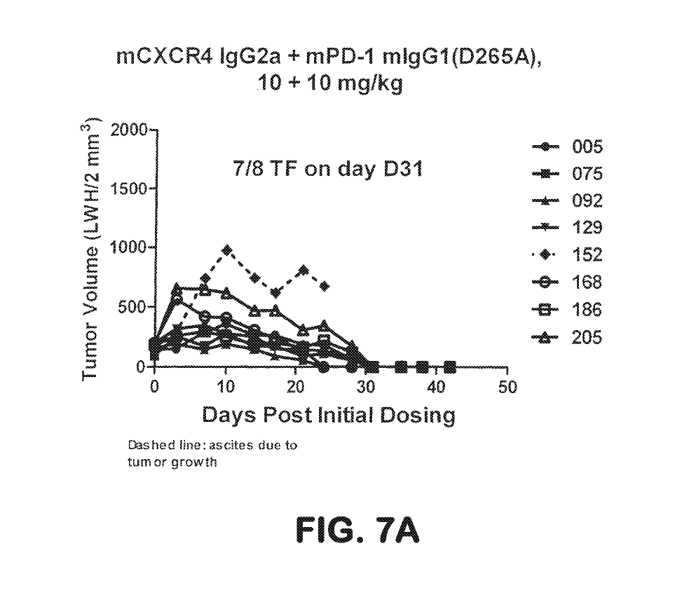

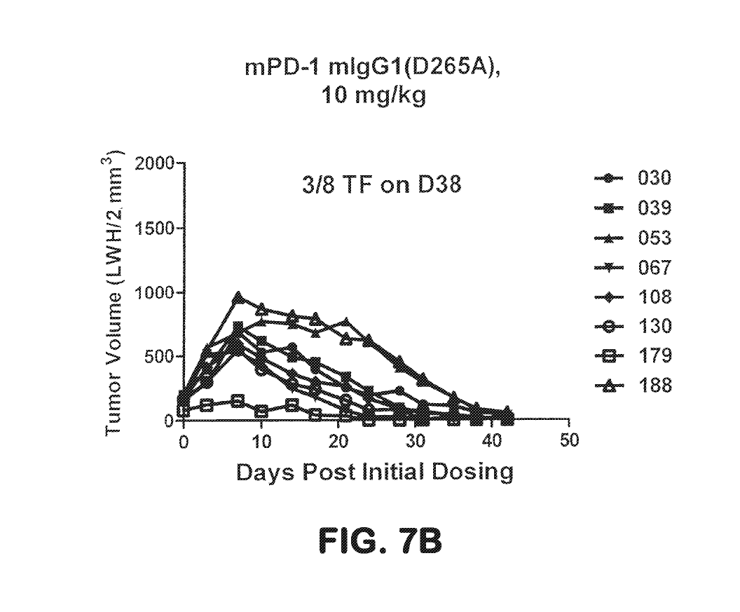

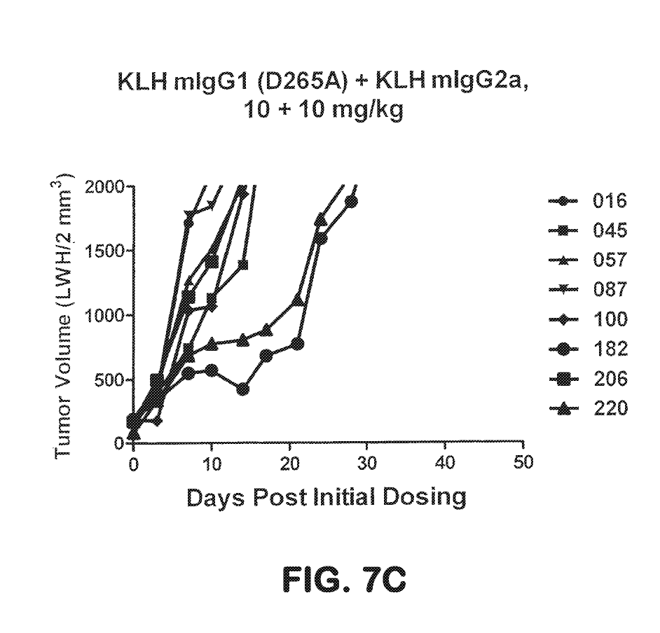

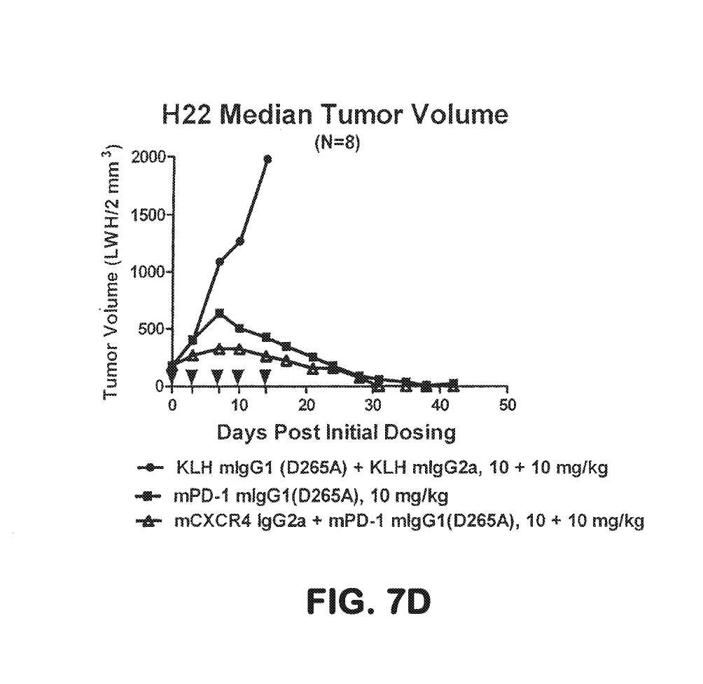

In the CXCR4-nonexpressing MC38 mouse model, low anti-tumor activity was observed with either of anti-CXCR4 IgG1 or anti-CXCR4 IgG2a (FIG. 6A), but potent activity was observed with both anti-CXCR4 isotype combinations (IgG1 or IgG2a) with anti-PD-1 (FIG. 6B), with the anti-CXCR4 IgG2a plus anti-PD1 being slightly more pronounced than the anti-CXCR4 IgG1 plus anti-PD-1. The combination of anti-CXCR4 IgG2a plus anti-PD1 also produced a robust anti-tumor effect in a H22 liver cancer model (Example 5; FIG. 7). The high level of synergism exhibited by the non-depleting anti-CXCR4 IgG1 with anti-PD-1 in a CXCR4⁻ tumor model suggests yet another possible mechanism of action. Blockade of the interaction between CXCR4 expressed on Tregs and/or MDSCs on the one hand and CXCL12 expressed in tumors on the other hand may decrease the trafficking of Tregs or MDSCs to the tumor microenvironment, thereby reducing the level of immune suppression.

Without being bound by any particular mechanism of action, these mouse data indicate that the combination of anti-PD-1 and anti-CXCR4 may be effective for treating various cancers, including SCLC, colon cancer and liver cancer. These data suggest that a depleting anti-CXCR4 Ab, for example an Ab having effector functions such as a human IgG1 or human IgG3 variant of ulocuplumab, may be highly effective in this combination.

Unmet Medical Need in PAC

Pancreatic cancer is the fourth most common cause of cancer-related death in the United States with a rising incidence during the past several decades. An estimated 48,960 people will be diagnosed with PAC, and approximately 40,560 will die of their disease (Siegel et al., 2015). The 1- and 5-year survival rates for newly diagnosed patients are 15% and less than 5%, respectively. If disease is diagnosed early (Stage I, Stage II), radical surgery with curative intent is the treatment goal (NCCN GUIDELINES®, Version 2.2015—Pancreatic Adenocarcinoma; Tempero et al., 2012). For patients with locally advanced or metastatic disease, the following systemic therapies have proven clinical benefit: FOLFIRINOX (a combination of folinic acid [FOL], fluorouracil [F], irinotecan [IRIN] and oxaliplatin [OX]), gemcitabine and the combination of gemcitabine plus albumin-bound paclitaxel. Phase 3 studies with gemcitabine demonstrated a median survival of 6.2 months and a 1-year survival rate of 20%. The Phase 3 PRODIGE trial comparing FOLFIRINOX to gemcitabine in metastatic patients with good performance status showed significant improvement in median PFS (6.4 vs. 3.3 months) and median OS (11.1 vs 6.8 months) with FOLFIRINOX compared to gemcitabine (Conroy et al., 2011). The Phase 3 IMPACT trial demonstrated improved PFS with the combination of gemcitabine/albumin-bound paclitaxel versus gemcitabine monotherapy (5.5 vs. 3.7 months) (Von Hoff et al., 2013). Second-line options in PAC include gemcitabine for patients that received FOLFIRINOX in the 1L and fluoropyrimidine-containing options for patients that received gemcitabine-based regimens in the 1L. However, no established standard of care exists for subjects who progress after 1L therapy in the advanced or metastatic disease setting.

Rationale for Combined Blockade of PD-1 and CXCR4 Signaling in PAC

Evidence supports targeting immune checkpoints in PAC due to the upregulation of the PD-1 pathway in pancreatic tumor biopsies and the correlation of PD-L1 expression with poor prognosis (Nomi et al., 2007). Similar to SCLC, pre-clinical evidence suggests that combination strategies with targeted agents may be required to overcome the refractory nature of the disease. For example, the combination of checkpoint inhibitors with therapies targeting the tumor microenvironment may allow for enhanced penetration of activated immune cells to the tumor site, thereby increasing tumor cell killing and prolonging survival. Pancreatic tumor biopsies express high levels of CXCR4 and this expression is associated with poor prognosis (Wang et al., 2013; Gao et al., 2010). CXCL12 promotes the growth of pancreatic tumor cells and is also reported to be an immunosuppressive component of the stromal microenvironment (Gao et al., 2010; Feig et al., 2013). In a pre-clinical mouse model of PAC, targeting the PD-1/PD-L1 pathway was only effective in the presence of concomitant inhibition of the CXCR4/CXCL12 pathway, further supporting this hypothesis (Feig et al., 2013; WO 2015/019284). It is, therefore, of interest to determine whether an anti-PD-1/anti-PD-L1 Ab such as nivolumab in conjunction with an anti-CXCR4/anti-CXCL12 Ab such as ulocuplumab provides an innovative combination regimen to improve response rates in PAC patients. Notably, the data obtained in mouse models of SCLC, colon cancer and liver cancer (Examples 2-5) show that an anti-CXCR4 Ab having effector functions, e.g., an IgG1 or IgG3 variant of ulocuplumab, may be more effective in synergizing with an anti-PD-1 Ab in inhibiting tumor growth.

Preclinical Rationale for the Dual Inhibition of CXCR4 and PD-1 Signaling

Pre-clinical xenograft tumor model studies were conducted with human cancer cell lines representing a number of hematologic malignancies including AML, MM and non-Hodgkin lymphomas (NHLs) such as CLL, FL, DLBCL and Burkitt's lymphoma, treated with ulocuplumab. Tumor growth inhibition was observed when ulocuplumab was administered as a single agent in these models (Kuhne et al., 2013; WO 2013/071068). In contrast, weak efficacy was observed with ulocuplumab monotherapy in solid tumor xenograft models including glioblastoma, melanoma, mesothelioma, pancreatic, breast carcinoma and SCLC (data not shown). In these solid tumor studies, tumor growth inhibition ranged from approximately 0-40% with the most convincing activity seen with the SCLC and triple negative breast carcinoma models.

Several malignancies present with tumors that contain fibroblast activating protein-positive (FAP⁺) carcinoma-associated fibroblasts that are major components of the tumor microenvironment. These malignancies express CXCL12 on the tumors and lack T-cells in the tumor nest (Fearon, 2014). These tumors, which tend to be refractory to standard treatments, include PAC, ovarian and colorectal cancer. Based on a PAC model, it was hypothesized that secretion of CXCL12 by FAP⁺ stromal cells resulted in CXCL12 binding to tumor cells, which interaction provided an immunosuppressive environment by inhibiting the recruitment of T-cells. This immunosuppression was overcome by using a combination of a small molecule CXCR4 antagonist and an anti-PD-L1 Ab, resulting in recruitment of CD3⁺ T-cells and significant tumor growth control (Feig et al., 2013; WO 2015/019284).

A similar finding and evidence for an important role for the CXCL12/CXCR4 pathway in immune surveillance was also recently reported using an orthotopic model of HCC (Chen et al., 2015). It was shown that sorafenib, the standard of care for HCC, induced hypoxia which led to upregulation of CXCL12 and PD-L1 expression by tumor cells. Following treatment with sorafenib, a small-molecule CXCR4 inhibitor and anti-PD-1 mAb resulted in reduced tumor growth and lung metastasis and increased CD8⁺ T-cell recruitment in tumors. It was concluded that blockade of CXCR4 and PD-1 pathway prevents suppression of immune cell function, increases recruitment of immune cells into the tumor and ultimately delays progression of HCC (Chen et al., 2015).

Collectively, the weak monotherapy activity observed with anti-CXCR4 in solid tumor animal models, and indications of a role of CXCR4/CXCL12 in immune surveillance with supportive in vivo efficacy data, provides a rationale for the testing of an anti-CXCR4 Ab (e.g., ulocuplumab) in combination with an anti-PD-1 Ab (e.g., nivolumab). The mouse data suggest that an IgG1 or IgG3 variant of ulocuplumab may be a batter choice for this study but such a variant is not yet available for clinical testing. However, surprising and unexpected complications have sometimes been observed when immunotherapeutics are combined with other anti-cancer agents. For example, 1L therapy of two melanoma patients carrying BRAF V600E mutations with anti-PD-1 agents (nivolumab and pembrolizumab, respectively) did not cause significant toxicity, but treatment with vemurafenib (ZELBORAF®) upon disease progression resulted in severe hypersensitivity drug eruptions with multi-organ injury early in their vemurafenib treatment course (Johnson et al, 2013). One patient subsequently developed acute inflammatory demyelinating polyneuropathy and the other developed anaphylaxis upon low-dose vemurafenib rechallenge.

Similarly, in a Phase 1 dose-escalation trial of the combination of sunitinib, an anti-angiogenic tyrosine kinase inhibitor, and tremelimumab, an anti-CTLA-4 Ab (Ribas, 2010; U.S. Pat. No. 6,682,736), in 28 subjects with metastatic RCC, an unexpected toxicity of rapid-onset renal failure was observed in 4 subjects out of 13 who received sunitinib 37.5 mg daily in combination with 10 mg/kg or 15 mg/kg tremelimumab once every 12 weeks, and one of these patients suffered a sudden death (Rini et al., 2011). Although a 43% partial response rate was observed, the toxicity of the combination at the maximal tolerated dose (MTD; sunitinib 37.5 mg daily plus tremelimumab 10 mg/kg q 12 weeks) was deemed unacceptable.

Thus, the combination of an immune checkpoint inhibitor drug such as an anti-PD-1/anti-PD-L1 Ab with another anti-cancer therapy such as an anti-CXCR4/anti-CXCL12 Ab is unpredictable. Despite a sound rationale for combining such drugs, it was not known prior to the studies described herein whether the combination of an anti-PD-1/anti-PD-L1 Ab and a CXCR4/CXCL12 Ab would be significantly more effective in treating refractory cancers in human subjects than treatment of these cancers with the individual agents.

Overall Risk/Benefit Assessment

There is very little treatment success for PAC patients failing 1L chemotherapy. Second-line treatment options include capecitabine and other chemotherapy-based options, none of which has demonstrated a survival benefit. Furthermore, no targeted agent has been approved for this disease in either newly diagnosed or refractory patient populations. For newly diagnosed SCLC patients, platinum-based chemotherapy is effective with significant response rates; however, most responses are not durable. Time to relapse after primary response to platinum-based agents is informative when determining the success rates to subsequent treatment options. For platinum-sensitive patients who have progressed, some responses can be seen after 2L chemotherapy but all patients eventually relapse. However, in platinum-refractory patients, very little success is anticipated when using a 2L chemotherapy agent, which represents a significant unmet need in this patient population. The refractory nature of PAC and SCLC may be a result, in part, of the immunosuppressive stromal microenvironment that prevents activated lymphocytes from infiltrating the tumor site. The combination of an anti-CXCR4/anti-CXCL12 Ab with an anti-PD-1/anti-PD-L1 Ab, as described herein, offers a unique opportunity to target both the stromal microenvironment and the activation of tumor-killing T cells. The ability of an anti-CXCR4/anti-CXCL12 Ab to increase the sensitivity of these tumor types to checkpoint inhibition with an anti-PD-1/anti-PD-L1 Ab may increase the treatment options in these refractory patient populations.

Ulocuplumab has demonstrated a manageable safety profile in two Phase 1 clinical trials in hematological malignancies (Becker et al., 2014; Ghobrial et al., 2014). Other therapeutic agents that target the CXCR4/CXCL12 pathway, including the approved drug plerixafor (AMD3100; MOZOBIL®), have demonstrated an acceptable toxicity profile in combination with background SOC in similar patient populations. In SCLC, a small peptide CXCR4 inhibitor was recently found to be safe and tolerable in a large, randomized Phase 2 SCLC trial (Spigal et al., 2014). While the safety of CXCR4 inhibition has been repeatedly demonstrated with multiple agents, limited clinical activity has been demonstrated. In both ulocuplumab trials in hematological indications, modest preliminary clinical activity was observed when combined with systemic chemotherapy or SOC. Other CXCR4 inhibitors have failed to meet primary endpoints in randomized controlled trials, and plerixafor has reported very limited efficacy data outside of the primary indication. However, there exists the potential that combination with other classes of targeted agents may enhance the activity of CXCR4 antagonists. The present disclosure relates to treatment of cancer patients with an anti-CXCR4 Ab such as ulocuplumab, or an anti-CXCL12 Ab such as 2A5 (U.S. Pat. No. 8,496,931) to block the immunosuppressive stromal microenvironment surrounding solid tumors as a means of enhancing the activity of an immune checkpoint inhibitor, specifically an anti-PD-1 Ab such as nivolumab, or an anti-PD-L1 Ab such as BMS-936559 (WO 2013/173223) and increasing tumor cell killing.

Nivolumab has demonstrated a manageable safety profile in more than 4000 patients in numerous early and late stage clinical trials. Preliminary data from a Phase 2 study of nivolumab monotherapy in SCLC and PAC show a similar toxicity profile compared to other solid tumor types. While there is clear benefit of nivolumab in many cancer patients, a significant proportion of patients fail to respond to monotherapy. Furthermore, there are some tumor types that have yet to show significant responses to checkpoint inhibition. The combination of nivolumab with agents that target the immunosuppressive microenvironment has the potential to benefit subjects with tumors that show low response to nivolumab monotherapy.

Broad Spectrum of Cancers Amenable to Treatment

Whereas the present disclosure exemplifies the treatment of SCLA and PAC by dual blockade of the PD-1 and CXCR4 signaling pathways, other cancers may be amendable to this combination therapy. For example, data reported by Chen et al. (2015) suggest that HCC may also be amenable to treatment. In addition, given the demonstrated efficacy of nivolumab a broad range of cancers, many other cancers may be treatable using the present combination of Abs. Thus, in certain embodiments, the disclosed combination therapy methods may be used to treat a cancer which is a solid tumor. In certain preferred embodiments, the solid tumor is SCLC or PAC. In other preferred embodiments, the solid tumor is HCC. In further embodiments, the solid tumor is a cancer selected from squamous cell carcinoma, non-small cell lung cancer, squamous non-small cell lung cancer (NSCLC), non squamous NSCLC, glioma, gastrointestinal cancer, renal cancer, ovarian cancer, liver cancer, colorectal cancer, endometrial cancer, kidney cancer, prostate cancer, thyroid cancer, neuroblastoma, glioblastoma, stomach cancer, bladder cancer, hepatoma, breast cancer, colon carcinoma, head and neck cancer, gastric cancer, germ cell tumor, pediatric sarcoma, sinonasal natural killer, melanoma, skin cancer, bone cancer, cervical cancer, uterine cancer, carcinoma of the fallopian tubes, carcinoma of the endometrium, carcinoma of the cervix, carcinoma of the vagina, carcinoma of the vulva, cancer of the anal region, testicular cancer, cancer of the esophagus, cancer of the small intestine, cancer of the endocrine system, cancer of the parathyroid gland, cancer of the adrenal gland, sarcoma of soft tissue, cancer of the urethra, cancer of the ureter, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, cancers of viral origin, and any combination of these cancers. In certain embodiments, the cancer is an advanced, unresectable, metastatic, refractory cancer, and/or recurrent cancer.

Both nivolumab and ulocuplumab have exhibited efficacy in early stage clinical trials in patients afflicted with hematological malignancies (Ansell et al., 2015; Becker et al., 2014; Ghobrial et al., 2014). Recently, it was demonstrated that CXCL12 from bone marow stroma, endothelium or osteoblasts promotes T cell acute lymphoblastic leukemia (T-ALL) survival while CXCR4 is required for T-ALL homing, and deletion of Cxcr4 or Cxcl12 genes or inhibition of CXCR4 with a small molecule antagonist in mouse models inhibited T-ALL progression (Pitt et al., 2015; Passaro et al., 2015). Thus, without being bound by any particular theory or mechanism of action, therapeutic methods disclosed herein combining blockade of the PD-1 and CXCR4 signaling pathways may also be used to treat hematological malignancies.

Hematological malignancies include liquid tumors derived from either of the two major blood cell lineages, i.e., the myeloid cell line (which produces granulocytes, erythrocytes, thrombocytes, macrophages and mast cells) or the lymphoid cell line (which produces B, T, NK and plasma cells), including all types of leukemias, lymphomas, and myelomas. Accordingly, hematological malignancies that may be treated using the present methods include, for example, cancers selected from acute, chronic, lymphocytic (lymphoblastic) and/or myelogenous leukemias, such as acute lymphoblastic leukemia (ALL), acute myelogenous leukemia (AML), chronic lymphocytic leukemia (CLL), and chronic myelogenous leukemia (CML); lymphomas, such as Hodgkin's lymphoma (HL; Hodgkin disease), non-Hodgkin's lymphomas (NHLs), of which about 85% are B cell lymphomas, including diffuse large B-cell lymphoma (DLBCL), follicular lymphoma (FL), chronic lymphocytic leukemia (CLL)/small lymphocytic lymphoma (SLL), mantle cell lymphoma, marginal zone B-cell lymphomas (mucosa-associated lymphoid tissue (MALT) lymphoma, nodal marginal zone B-cell lymphoma, and splenic marginal zone B-cell lymphoma), Burkitt's lymphoma, lymphoplasmacytoid lymphoma (LPL; also known as Waldenstrom's macroglobulinemia (WM)), hairy cell lymphoma, and primary central nervous system (CNS) lymphoma, NHLs that are T cell lymphomas, including precursor T-lymphoblastic lymphoma/leukemia, T-lymphoblastic lymphoma/leukemia (T-Lbly/T-ALL), peripheral T-cell lymphomas such as cutaneous T-cell lymphoma (CTLC, i.e., mycosis fungoides, Sezary syndrome and others), adult T-cell lymphoma/leukemia, angioimmunoblastic T-cell lymphoma, extranodal natural killer/T-cell lymphoma nasal type, enteropathy-associated intestinal T-cell lymphoma (EATL), anaplastic large-cell lymphoma (ALCL), and peripheral T-cell lymphoma unspecified, acute myeloid lymphoma, lymphoplasmacytoid lymphoma, monocytoid B cell lymphoma, angiocentric lymphoma, intestinal T-cell lymphoma, primary mediastinal B-cell lymphoma, post-transplantation lymphoproliferative disorder, true histiocytic lymphoma, primary effusion lymphoma, diffuse histiocytic lymphoma (DHL), immunoblastic large cell lymphoma, and precursor B-lymphoblastic lymphoma; myelomas, such as multiple myeloma, smoldering myeloma (also called indolent myeloma), monoclonal gammopathy of undetermined significance (MGUS), solitary plasmocytoma, IgG myeloma, light chain myeloma, nonsecretory myeloma, and amyloidosis; and any combinations of said hematological malignancies. The present methods are also applicable to treatment of advanced, metastatic, refractory and/or recurrent hematological malignancies.

Rationale for Study Design

The clinical study disclosed herein is a Phase 1/2 open-label study of ulocuplumab combined with nivolumab to estimate the safety and efficacy in subjects with SCLC and PAC. Since this is the first time evaluating ulocuplumab in solid tumors, a dose limiting toxicity (DLT) evaluation period is conducted for the first 3-6 subjects at each dose (400 mg, 800 mg and 1600 mg weekly ulocuplumab combined with nivolumab). For the sentinel dose level (400 mg weekly ulocuplumab), both tumor types are combined for the safety evaluation. For the 800 mg and 1600 mg weekly ulocuplumab dose levels, each tumor type is evaluated for safety independently in the event that tumor specific AE may emerge. A Rolling-6 design is utilized for the DLT evaluation period, which allows for a range of 3-6 evaluable subjects to contribute to the DLT evaluation depending on how many subjects are enrolled and still being evaluated during the DLT period (Skolnik et al., 2007). This design is particularly useful in SCLC and PAC, where subjects often discontinue due to disease progression prior to completion of the DLT period.

After completion of the DLT period, the Dose Evaluation Phase simultaneously evaluates two different ulocuplumab doses (800 and 1600 mg) and two different ulocuplumab schedules for 1600 mg (weekly and every 2 weeks). A recommended dose and schedule is selected for the Dose Expansion Phase and is based on the totality of safety and efficacy data across three cohorts, within each tumor type. The level of efficacy at the recommended dose also dictates the type of study design selected for the Dose Expansion Phase. One option is to continue with a single arm study at the recommended dose level if only moderate efficacy is observed during the Dose Evaluation Phase. However, if substantial efficacy is observed, a randomized Phase 2 design with a comparative arm is initiated. This adaptive approach allows for the rapid implementation of confirmatory efficacy studies and proactively plans for performing the most informative studies with this combination regimen for these advanced tumor types.

### Pharmaceutical Compositions and Dosage Regimens

Abs used in the methods disclosed herein may be constituted in a composition, e.g., a pharmaceutical composition containing an Ab and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an Ab is suitable for intravenous, intramuscular, subcutaneous, parenteral, spinal or epidermal administration (e.g., by injection or infusion). A pharmaceutical composition of the invention may include one or more pharmaceutically acceptable salts, anti-oxidants, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. For administration of an anti-PD-1, anti-PD-L1, anti-CXCR4 Ab or anti-CXCL12, including for combination use, the dosage may range from about 0.01 to about 20 mg/kg, preferably from about 0.1 to about 15 mg/kg, of the subject's body weight. For example, dosages can be about 0.1, 0.3, 1, 2, 3, 5, 10 or 15 mg/kg body weight, and more preferably, about 0.3, 1, 3, or 10 mg/kg body weight. Alternatively, a fixed or flat dose, e.g., about 50-2000 mg of the Ab, instead of a dose based on body weight, may be administered weekly or once every two weeks. The dosing schedule is typically designed to achieve exposures that result in sustained receptor occupancy (RO) based on typical pharmacokinetic properties of an Ab. An exemplary treatment regime entails administration once per week, once every 2 weeks, once every 3 weeks, once every 4 weeks, once a month, once every 3-6 months or longer. In certain preferred embodiments, the anti-PD-1 or anti-PD-L1 Ab is administered to the subject once every 2 weeks. In other preferred embodiments, the Ab is administered once every 3 weeks. The dosage and scheduling may change during a course of treatment.

When used in combinations, a subtherapeutic dosage of one or both Abs, e.g., a dosage of an anti-PD-1, anti-PD-L1, anti-CXCR4 and/or anti-CXCL12 Ab lower than the typical or approved monotherapy dose, may be used. For example, a dosage of nivolumab that is significantly lower than the approved 3 mg/kg every 2 weeks, for instance, 1.0 mg/kg or less every 3 or 4 weeks, is regarded as a subtherapeutic dosage. RO data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al., 2010). Thus, 0.3 mg/kg dosing may allow for sufficient exposure to lead to significant biologic activity.

A synergistic interaction between the anti-PD-1/anti-PD-L1 and anti-CXCR4/anti-CXCL12 Abs favors the administration of one or both of these therapeutics to a patient at subtherapeutic dosages, i.e., a dose of the therapeutic agent that is significantly lower than the typical or approved dose when administered as monotherapy for the treatment of the cancer. In certain embodiments of the disclosed combination therapy methods, the anti-PD-1/anti-PD-L1 Ab or antigen-binding portion thereof is administered to a cancer patient at a subtherapeutic dose. In other embodiments, the anti-CXCR4/anti-CXCL12 Ab is administered at a subtherapeutic dose. In further embodiments, the anti-PD-1/anti-PD-L1 and anti-CXCR4/anti-CXCL12 Abs or antigen-binding portions thereof are each administered to the patient at a subtherapeutic dose.

The administration of such a subtherapeutic dose of one or both Abs may reduce adverse events compared to the use of higher doses of the individual Abs in monotherapy. Thus, the success of the disclosed methods of combination therapy may be measured not only in improved efficacy of the combination of Abs relative to monotherapy with these Abs, but also in increased safety, i.e., a reduced incidence of adverse events, from the use of lower dosages of the drugs in combination relative to the monotherapy doses.

Dose Selection of Nivolumab

For nivolumab, a dosage of 3 mg/kg every 2 weeks has been determined to be safe and tolerable as a monotherapy in multiple solid tumor programs and is the approved dosage in melanoma and NSCLC. This dosage is being evaluated in multiple clinical studies in various hematological malignancies and other solid tumors, including PAC and SCLC, and has not demonstrated any dose-related toxicity. Nivolumab has also been evaluated with various combination partners at this dosage and has not revealed any unexpected safety concerns. Therefore, the dosage of 3 mg/kg every 2 weeks is expected to be tolerable as a combination partner with an anti-CXCR4 or anti-CXCL12 Ab.

Dose Selection of Ulocuplumab

Ulocuplumab has been evaluated in over 140 subjects across various hematological malignancies at dose levels ranging from 0.3 mg/kg to 10 mg/kg with a safe and tolerable profile. The majority of subjects have received the 10 mg/kg dose and no exposure-related AEs have been observed. No MTD was identified. Results from a preliminary population PK analysis have suggested that body weight had only modest effects on the disposition of ulocuplumab. The allometric coefficients of baseline body weight for ulocuplumab clearance (CL) and central volume of distribution (Vc) were estimated to be 0.33 and 0.41, respectively. It has been reported that a flat dosing regimen may provide more uniform exposures when the estimated allometric exponent of body weight on CL and Vc in the population PK model are less than 0.5 (Bai et al., 2012). This information supports a flat-dose schedule, as opposed to body weight-normalized dosing. Simulations based on the population PK model indicated that a dose of 800 mg weekly (equivalent to 10 mg/kg weekly for an 80-kg subject) would provide exposures largely within the concentration ranges observed in subjects who received 10 mg/kg ulocuplumab in the two Phase 1 studies (Becker et al., 2014; Ghobrial et al., 2014).

Peripheral ulocuplumab CXCR4 RO was measured in subjects with AML and the exposure-RO analysis showed that high RO was achieved over much of concentrations following the 10-mg/kg dose. Correspondingly, simulations based on the population PK and exposure-RO models have suggested that ulocuplumab exposures following the 800-mg weekly dose would also provide high median RO (greater than 90%) in the tumor tissues throughout the dosing period and, therefore, is expected to be an efficacious dose.

Dosage Regimens Employed in the Present Methods

In certain embodiments of the disclosed methods, the anti-PD-1 or anti-PD-L1 Ab or antigen-binding portion thereof is administered to the subject at a dose ranging from about 0.1 to about 20.0 mg/kg body weight once every 2, 3 or 4 weeks. In certain preferred embodiments, the anti-PD-1 Ab or antigen-binding portion thereof is administered at a dose of about 2 or about 3 mg/kg body weight once every 2 or 3 weeks, whereas the anti-PD-L1 Ab or antigen-binding portion thereof is administered at a dose of about 10 or about 15 mg/kg body weight once every 2 or 3 weeks. In certain embodiments of the methods employing nivolumab, this Ab is administered at the approved dose of 3 mg/kg every 2 weeks. Similarly, in certain embodiments employing pembrolizumab, this Ab is administered at the approved dose of 2 mg/kg every 3 weeks.

In certain embodiments of the present methods, the anti-CXCR4 or anti-CXCL12 Ab or antigen-binding portion thereof is administered to the subject at a at a flat dose of about 50-2000 mg weekly. In certain other embodiments, the anti-CXCR4 Ab or anti-CXCL12 or antigen-binding portion thereof is administered at a flat dose of about 200, about 400, about 800, or about 1600 mg weekly. In certain preferred embodiments, the anti-CXCR4 Ab or anti-CXCL12 or antigen-binding portion thereof is administered at a flat dose of about 400 or about 800 mg weekly. In certain other embodiments, the anti-CXCR4 Ab or anti-CXCL12 or antigen-binding portion thereof is administered at a flat dose of about 1600 mg once every 2 weeks.

In further embodiments, the anti-CXCR4/anti-CXCL12 Ab or antigen-binding portion thereof is administered at a dose ranging from about 0.1 to about 20.0 mg/kg body weight once every 2, 3 or 4 weeks. In certain preferred embodiments, the anti-CXCR4/anti-CXCL12 Ab or antigen-binding portion thereof is administered at a dose of about 3 or about 10 mg/kg body weight once every 2 or 3 weeks.

Although there is no evidence to suggest that the combination of nivolumab and ulocuplumab in the combination clinical study described herein would result in overlapping or synergistic toxicities, given the first-in-human nature of this combination, dosing is initiated for ulocuplumab at 400 mg weekly (i.e., half of the highest tolerated dose to date of 800 mg weekly). In the current study, following a safety evaluation period for the 400 mg weekly starting dose, in the event of dose limiting toxicity (DLT), a lower dose of 200 mg weekly is also evaluated. Conversely, in the event the 400 mg weekly dose is deemed to be safe and tolerable, higher dose levels of 800 mg weekly and 1600 mg weekly are also evaluated sequentially. The 1600 mg weekly dose, based on the current model and sensitivity analysis, is expected to provide sustained, near maximum RO in the majority of the subjects. In addition, in the event the 1600 mg weekly dose is determined to be safe following the DLT period, a regimen comprising 1600 mg administered every 2 weeks is evaluated. Administration of ulocuplumab once every 2 weeks aligns with the dosing schedule for nivolumab and would allow for improved patient convenience.

Accordingly, certain embodiments of the present combination therapy methods comprise administering to the subject a combination of: (a) an Ab or an antigen-binding portion thereof that binds to PD-1 and inhibits PD-1/PD-L1 signaling, wherein the anti-PD-1 Ab or portion thereof is administered at a dose of about 2 or about 3 mg/kg body weight once every 2 or 3 weeks; and (b) an Ab or an antigen-binding portion thereof that binds to CXCR4 and inhibits CXCR4/CXCL12 signaling, wherein the anti-CXCR4 Ab or portion thereof is administered at a flat dose of about 400 or about 800 mg weekly. In certain preferred embodiments, the anti-PD-1 Ab is nivolumab which is administered at a dose of about 3 mg/kg body weight once every 2 weeks, and the anti-CXCR4 Ab is ulocuplumab which is administered at a flat dose of about 400-800 mg weekly. In certain other embodiments, the anti-PD-1 Ab is pembrolizumab which is administered at a dose of about 2 mg/kg body weight once every 3 weeks, and the anti-CXCR4 Ab is ulocuplumab which is administered at a flat dose of about 400-800 mg weekly.

In certain embodiments of any of the methods disclosed herein, the anti-PD-1, anti-PD-L1, anti-CXCR4 and/or anti-CXCL12 Abs are formulated for intravenous administration. In certain embodiments, the anti-PD-1/anti-PD-L1 Ab or antigen-binding portion thereof and the anti-CXCR4/anti-CXCL12 Ab or antigen-binding portion thereof are administered sequentially to the subject. “Sequential” administration means that one of the anti-PD-1/anti-PD-L1 and anti-CXCR4/anti-CXCL12 Abs is administered before the other. Typically, the Ab administered second is administered while the activity of the first-administered Ab is ongoing in the subject. Either Ab may be administered first; i.e., in certain embodiments, the anti-PD-1/anti-PD-L1 Ab is administered before the anti-CXCR4/anti-CXCL12 Ab, whereas in other embodiments, the anti-CXCR4/anti-CXCL12 is administered before the anti-PD-1/anti-PD-L1 Ab. Typically, each Ab is administered by intravenous infusion over a period of about 60 minutes.

In certain embodiments of sequential administration, for the convenience of the patient, the anti-PD-1/anti-PD-L1 and anti-CXCR4/anti-CXCL12 Abs or portions thereof are administered within 30 minutes of each other. Typically, when both the anti-PD-1/anti-PD-L1 and anti-CXCR4/anti-CXCL12 Abs are to be administered on the same day, separate infusion bags and filters are used for each infusion. After the administration of the first Ab, say, ulocuplumab, the ulocuplumab infusion is promptly followed by a saline flush to clear the line of ulocuplumab before starting the infusion of the second Ab, e.g., nivolumab. In other embodiments, the two Abs are administered within 1, 2, 4, 8, 24 or 48 hours of each other.

Because checkpoint inhibitor Abs have been shown to produce very durable responses, in part due to the memory component of the immune system (see, e.g., WO 2013/173223; Lipson et al., 2013; Wolchok et al., 2013), the activity of an administered anti-PD-1/anti-PD-L1 Ab may be ongoing for several weeks, several months, or even several years. In certain embodiments, the present combination therapy methods involving sequential administration entail administration of an anti-CXCR4/anti-CXCL12 Ab to a patient who has been previously treated with an anti-PD-1/anti-PD-L1 Ab. In further embodiments, the anti-CXCR4/anti-CXCL12 Ab is administered to a patient who has been previously treated with, and progressed on, an anti-PD-1/anti-PD-L1 Ab. In other embodiments, the present combination therapy methods involving sequential administration entail administration of an anti-PD-1/anti-PD-L1 Ab to a patient who has been previously treated with an anti-CXCR4/anti-CXCL12 Ab, optionally a patient whose cancer has progressed after treatment with the anti-CXCR4/anti-CXCL12 Ab.

In certain other embodiments, the anti-PD-1/anti-PD-L1 and anti-CXCR4/anti-CXCL12 Abs are administered concurrently, either admixed as a single composition in a pharmaceutically acceptable formulation for concurrent administration, or concurrently as separate compositions with each Ab in formulated in a pharmaceutically acceptable composition.

Factors Affecting Dosing Regimens

Dosage and frequency vary depending on the half-life of the Ab in the subject. In general, human Abs show the longest half-life, followed by humanized Abs, chimeric Abs, and nonhuman Abs. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being unduly toxic to the patient. The selected dosage level depends upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Methods of Reducing Adverse Events

In certain embodiments of the present methods, the anti-PD-1/anti-PD-L1 Ab or antigen-binding portion thereof is administered at a subtherapeutic dose. In certain other embodiments, the anti-CXCR4/anti-CXCL12 Ab or antigen-binding portion is administered at a subtherapeutic dose. In further embodiments, the anti-PD-1/anti-PD-L1 Ab or antigen-binding portion thereof and the anti-CXCR4/anti-CXCL12 Ab or antigen-binding portion thereof are each administered at a subtherapeutic dose. The administration of at least one of the Abs at a subtherapeutic dose may reduce adverse events in the subject, for example, compared to the incidence of adverse events when the Ab is administered at its typical or approved dose in monotherapy. Accordingly, this disclosure provides a method for treating a subject afflicted with a cancer comprising administering to the subject a combination of: (a) an Ab or an antigen-binding portion thereof that disrupts the interaction between PD-1 and PD-L1 and inhibits PD-1/PD-L1 signaling; and (b) an Ab or an antigen-binding portion thereof that disrupts the interaction between CXCR4 and CXCL12 and inhibits CXCR4/CXCL12 signaling, wherein at least one of the Abs or portions thereof is administered at a subtherapeutic dose, which subtherapeutic dose or doses reduces adverse events in the subject.

The disclosure also provides a method for reducing adverse events in a subject undergoing treatment for cancer comprising administering to the subject a combination of: (a) an Ab or an antigen-binding portion thereof that disrupts the interaction between PD-1 and PD-L1 and inhibits PD-1/PD-L1 signaling; and (b) an Ab or an antigen-binding portion thereof that disrupts the interaction between CXCR4 and CXCL12 and inhibits CXCR4/CXCL12 signaling, wherein at least one of the Abs or portions thereof is administered at a subtherapeutic dose.

In certain embodiments of any of the therapeutic methods disclosed herein, administration of the combination of Abs is continued for as long as clinical benefit is observed or until unmanageable toxicity or disease progression occurs.

### Medical Uses of Anti-PD-1/Anti-PD-L1 and Anti-CXCR4/Anti-CXCL12 Abs

This disclosure also provides an anti-PD-1/anti-PD-L1 Ab or an antigen-binding portion thereof and an anti-CXCR4/anti-CXCL12 Ab or an antigen-binding portion thereof for use in combination in treating a subject afflicted with cancer comprising dual inhibition of the PD-1/PD-L1 and CXCR4/CXCL12 signaling pathway. These Abs may be used in combination therapy of the full range of cancers disclosed herein. In certain preferred embodiments, the cancer is SCLC. In other preferred embodiments, the cancer is PAC. In yet other preferred embodiments, the cancer is HCC.

One aspect of the disclosed invention is the combined use of an anti-PD-1/anti-PD-L1 Ab or an antigen-binding portion thereof and an anti-CXCR4/anti-CXCL12 Ab or an antigen-binding portion thereof for the preparation of a medicament for treating a subject afflicted with a cancer. Uses of any such anti-PD-1/anti-PD-L1 Ab and anti-CXCR4/anti-CXCL12 Ab in combination for the preparation of medicaments are broadly applicable to the full range of cancers disclosed herein. In certain preferred embodiments of these uses, the cancers are SCLC, PAC and HCC.

This disclosure also provides medical uses of an anti-PD-1/anti-PD-L1 Ab in combination with an anti-CXCR4/anti-CXCL12 Ab corresponding to all the embodiments of the methods of treatment employing this combination of Abs described herein.

### Kits

Also within the scope of the present invention are kits comprising an anti-PD-1/anti-PD-L1 Ab and an anti-CXCR4/anti-CXCL12 Ab for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) one or more dosages ranging from about 0.1 to about 20 mg/kg body weight of an Ab or an antigen-binding portion thereof that disrupts the interaction between PD-1 and PD-L1 and inhibits PD-1/PD-L1 signaling; (b) one or more dosages ranging from about 400 to about 800 mg of an Ab or an antigen-binding portion thereof that disrupts the interaction between CXCR4 and CXCL12 and inhibits CXCR4/CXCL12 signaling; and (c) instructions for using the Ab or portion thereof that inhibits PD-1/PD-L1 signaling and the Ab or portion thereof that inhibits CXCR4/CXCL12 signaling in any of the combination therapy methods disclosed herein. In certain embodiments, the Abs may be co-packaged in unit dosage form. In certain preferred embodiments for treating human patients, the kit comprises an anti-human PD-1 Ab disclosed herein, e.g., nivolumab or pembrolizumab. In other preferred embodiments, the kit comprises an anti-human CXCR4 Ab disclosed herein, e.g., ulocuplumab.

The present invention is further illustrated by the following examples which should not be construed as further limiting. The contents of all references cited throughout this application are expressly incorporated herein by reference.

### Example 1

### Use of Syngeneic Mouse Tumor Models to Study Anti-Tumor Activity of Antibodies

### Tumor Efficacy Studies in Kp1, Kp3 and MC38 Mice

The Kp1 and Kp3 cell lines were derived from SCLC-like lung tumors of transgenic mice in which three oncogenes, p53, Rb and p130, had been inactivated (Schaffer et al., 2010; Jahchan et al., 2013). The Kp1 and Kp3 mouse SCLC cell lines (Jahchan et al., 2013) were kindly provided by Dr. Julien Sage of Stanford University.

Mouse cell lines Kp1 (SCLC), Kp3 (SCLC) or MC38 (a mouse colon carcinoma cell derived from C57BL6/J mice) were cultured in Dulbecco's modified Eagle's medium (DMEM) (Corning Life Sciences, Manassas, Va.) supplemented with 10% fetal bovine serum. Cells were maintained in a humidified atmosphere at 37° C. and 5% CO₂. All cell lines were harvested in their exponential growth phase, and the cell number and viability assessed using a Cedex automated cell counter (Roche Diagnostics, Indianapolis, Ind.). All cell lines for in vivo studies were confirmed to be free of mycoplasma and rodent viral pathogens (IMPACT test).

For tumor studies, 5×10⁶ cells were implanted subcutaneously (s.c.) with 50% MATRIGEL™ (Becton Dickinson, San Jose, Calif.) into the flank of either B6129S1/J F1 (Kp1 or Kp3) or C57B16 mice (MC38). Mice were randomized into cohorts (typically 6-10 mice/group) when tumors reached a median size of approximately 25-50 mm³. All test agents (single agents or combinations) were administered intraperitoneally (i.p.) at doses and schedules indicated in the Figures. Tumor volumes, body weights and clinical observations were noted to establish efficacy and tolerability of test agents. Tumor caliper measurements were converted into tumor volumes using the formula: volume=1/2 (length×width×height). Tumor growth and body weight were monitored for up to 47 days after initial dosing.

On study, mice received sterile rodent chow and water ad libitum and were housed in sterile filter-top cages with 12-h light/dark cycles. All experiments were conducted in accordance with the guidelines of the Association for Assessment and Accreditation of Laboratory Animal Care International.

### Tumor Efficacy Studies in H22 Mice

The H22 (liver cancer) mouse cell line was maintained in vitro in RPMI-1640 medium (Corning Life Sciences) supplemented with 10% fetal bovine serum. The tumor cells were routinely sub-cultured twice weekly. Cells were harvested in their exponential growth phase and counted for tumor inoculation. Each mouse was inoculated s.c. at the right lower flank region with 2×10⁶ H22 tumor cells in 0.1 ml of PBS for tumor development. Mice were randomized into cohorts of 8 mice/group when tumors reached a mean size of about 169 mm³, and test agents (single agents or combinations) were administered i.p. to the tumor-bearing mice twice a week for five doses with the date of the first dosing denoted as Day 0. The isotype control group was treated with mouse IgG2a plus mouse IgG1D265A (a non-FcγR-binding mutant IgG1 isotype containing a D265A mutation; Clynes et al., 2000) each at 10 mg/kg. The PD-1 group was treated with anti-mouse PD-1 mouse IgG1D265A at 10 mg/kg. The CXCR4 and PD-1 combination group was treated with anti-mouse CXCR4 mIgG2a and anti-mouse PD-1 IgG1D265A each at 10 mg/kg. Tumor growth and body weight were monitored for 42 days after initial dosing.

### Flow Cytometry

Cell lines (KP cells, MC38) were harvested during their exponential growth phase, and cell number and viability were assessed using a Cedex automated cell counter. For FACS analysis, cells (10⁶ per well) were transferred into an U-Bottom plate (Polystyrene 96-well plate, Falcon REF#351177). Cells were washed with 200 μl of FACS buffer (PBS, 2% FBS, 0.1% NaN3) and centrifuged at 2,000 rpm for 1 min. Cells were Fc-blocked for 10 min on ice with purified rat anti-mouse CD16/CD32 (Mouse BD Fc block; BD Cat. No. 553142 (10 ug/ml)). CXCR4 immunostaining was conducted with Abs for mouse CXCR4 (anti-mouse CXCR4 PE, R&D Cat. No. FAB21651P) or isotype (rat IgG2b isotype control PE, R&D Cat. No. 1C013P) and live/dead stain (Aqua fluorescent reactive dye, Life Science Cat. No. L34957) (1:500). Staining was conducted for 30 min in the dark on ice (in 100 μl per well). Cells were then washed twice with FACS buffer as previously described. Cells were fixed with 4% PFA for 30 min on ice, followed by an additional wash with 200 μl of FACS buffer (2,000 rpm for 1 min). The cells were then resuspended in 150 μl of FACS buffer prior to either FACS Array or BD Canto II analysis. Data analysis was conducted using flowjo software.

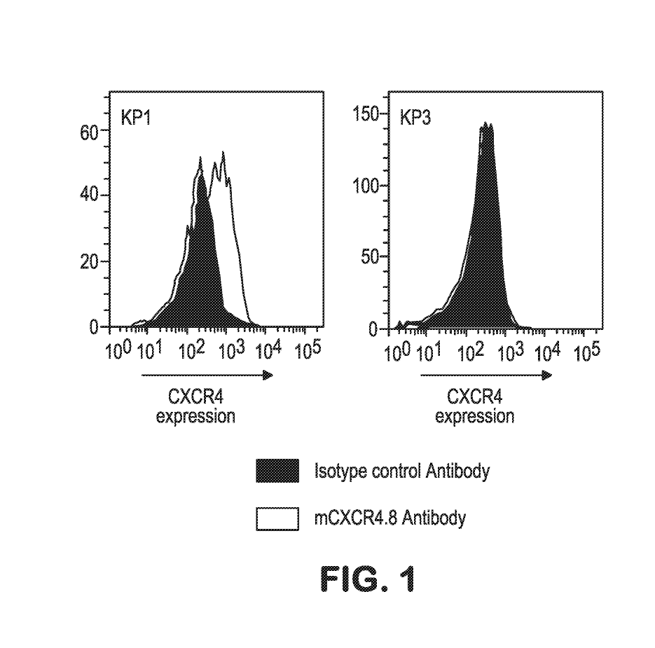

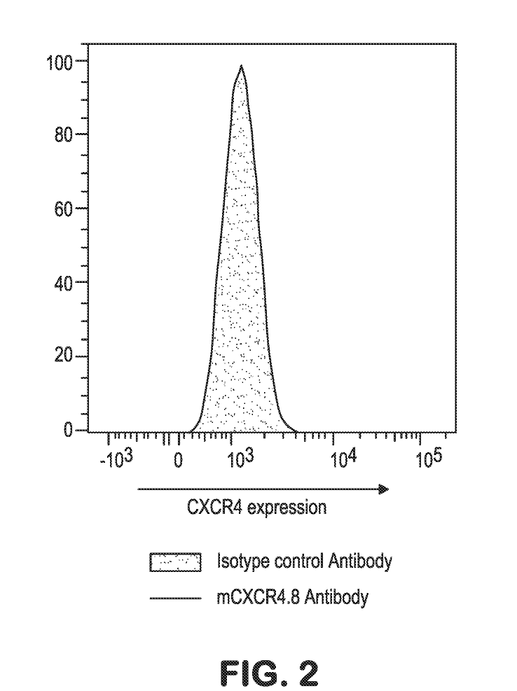

The expression of CXCR4 on the Kp1, Kp3 and MC38 cell lines was assessed by flow cytometry. As shown in FIG. 1, the Kp1 cell line expresses CXCR4 on the cell surface whereas and Kp3 shows no surface expression of CXCR4. FIG. 2 shows that the MC38 cell line also does not express CXCR4 on the cell surface. The expression of CXCR4 on various types of human T cells was also measured by flow cytometry. As shown in FIG. 3, human Tregs express considerably higher levels of CXCR4 than CD8+ T cells and T effector cells.

### Example 2

### Anti-Tumor Activity of Anti-CXCR4 in Combination with Anti-Pd-1 in CXCR4-Expressing Mouse Kp1 Tumor Model

The anti-tumor activity of an anti-mouse CXCR4 Ab was assessed, either alone or in combination with an anti-mouse PD-1 Ab, in the Kp1 CXCR4⁺ mouse SCLC model as described in Example 1.

The CXCR4 Ab used in this and the subsequent Examples was a mouse anti-mouse CXCR4 mAb, clone 4.8, constructed from a rat IgG2b anti-mouse CXCR4 mAb (Clone #247506; Cat. No. MAB21651; R&D Systems, Minneapolis, Minn.) in which the Fc portion was replaced with an Fc portion from a mouse IgG1 or mouse IgG2a isotype. The mIgG1 format of the anti-mCXCR4 mAb was intended to mimic the non-depleting biological properties of ulocuplumab which has a human IgG4 isotype, while the mIgG2a format (corresponding to human IgG1) was designed for potentially mediating depletion of cells to which the mAb binds.

The PD-1 Ab used in the Examples was mAb 4H2 with an engineered IgG1D265A isotype. Mab 4H2 is a chimeric rat-mouse anti-mPD-1 mAb constructed from a rat IgG2a anti-mouse PD-1 Ab in which the Fc portion was replaced with an Fc portion from a mouse IgG1 isotype (WO 2006/121168). In the mouse tumor experiments described herein that employed anti-mouse PD1, mAb 4H2 comprising the mIgG1D265A Fc portion was used. 4H2-mIgG1D265A has been shown to block binding of mPD-L1 and mPD-L2 to mPD-1, stimulate a T cell response, and exhibit the strongest inhibitory effect on MC38 tumor growth compared to the other mouse isotypes (WO 2006/121168).

The changes in median tumor volumes of the mice are plotted in FIGS. 4A and 4B. The anti-CXCR4 mIgG1 isotype shows practically no inhibition of tumor growth in this model system, with the median tumor volumes being similar to those in mice treated with mouse anti-Keyhole Limpet Hemocyanin (KLH) IgG1 mAb and vehicle (saline) negative controls (FIG. 4A), whereas the mIgG2a isotype of the anti-CXCR4 Ab exhibits the most robust inhibitory effect on Kp1 tumor growth (FIG. 4A). Anti-PD1 (mAb 4H2 mIgG1) shows a low level of anti-tumor activity (FIG. 4A).

When combined with anti-PD-1, the anti-CXCR4 IgG1 Ab showed a low degree of tumor inhibition compared to the controls (FIG. 4B). In contrast, the combination of the anti-CXCR4 IgG2a mAb with anti-PD-1 produced essentially total inhibition of tumor growth throughout the monitoring period (FIG. 4B). Thus, in this Kp1 CXCR4-expressing model, the combination of anti-CXCR4-IgG2a and anti-PD1 shows a strong synergistic effect in inhibiting growth of mouse SCLS tumor cells whereas the combination of anti-CXCR4-IgG1 and anti-PD1 did not significantly enhance the low level of anti-tumor activity of anti-PD-1 in this murine model (FIGS. 4A and 4B). A combination of Abs is considered synergistic if the antitumor effect of the combination is greater than the effect observed with monotherapy with the more efficacious Ab or greater than the sum of the level of inhibition exhibited by each Ab individually.

### Example 3

### Anti-Tumor Activity of Anti-CXCR4 in Combination with Anti-Pd-1 in CXCR4-Nonexpressing Mouse Kp3 Tumor Model

The anti-tumor activity of different isotypes of the anti-mouse CXCR4 Ab was assessed, either alone or in combination with anti-mouse PD-1, in a CXCR4⁻ Kp3 mouse SCLC tumor model as described in Example 1. A non-fucosylated (nf) anti-diphtheria toxin (DT) Ab with a human IgG1 Fc region, the anti-KLH IgG1 and anti-KLH IgG2a mAbs (simply designated “IgG1” or “IgG2a” in FIG. 5) were used as non-binding control Abs. The nf modification typically enhances ADCC activity.

In this experiment, a low level of tumor growth inhibition was observed with multiple non-binding control Abs compared to saline “vehicle”). See FIG. 5. The results for the controls Abs and single agents (anti-CXCR4 or anti-PD-1) are shown in FIG. 5A. This figure illustrates that anti-CXCR4 mIgG2a administered as a single agent exhibits appreciable anti-tumor activity, more than the level seen with anti-PD-1, despite the lack of expression of CXCR4 on Kp3 cells. FIG. 5B shows the effects of treatment with the combination of anti-CXCR4 and anti-PD-1 in the same experiment. It is evident that in this Kp3 tumor cell model that is relatively refractory to anti-PD1 treatment, there is still a modest enhancement in the level of anti-tumor activity with the anti-CXCR4 IgG2a plus anti-PD1 combination treatment compared to treatment with anti-CXCR4 IgG2a or anti-PD-1 alone (FIG. 5B). The lack of CXCR4 expression by tumor cells in this Kp3 model suggests that anti-CXCR4 may act on CXCR4-expressing targets other than the tumor itself, for example, Tregs and/or MDSCs. Blockade of the interaction between CXCR4 expressed on Tregs or MDSCs and CXCL12 expressed in tumors may decrease the recruitment of Tregs or MDSCs to the tumor, reducing the level of immune suppression. Binding of anti-CXCR4 IgG2a to CXCR4 on Tregs and/or MDSCs may also result in apoptosis and ADCC-, ADCP- and/or CDC-mediated depletion of these immunosuppressant cells, thereby enhancing the anti-tumor response of anti-PD-1.

The present results contrast with the data shown in FIG. 4B, where a strong synergistic interaction, evidenced by a massive enhancement of anti-tumor activity, was seen between anti-CXCR4 IgG2a and anti-PD1 in the CXCR4⁺ Kp1 tumor model. This suggests that in the Kp1 model additional mechanisms of anti-CXCR4 action may be involved. For example, CXCR4-expressing tumor cells may be destroyed by anti-CXCR4 IgG2a directly by apoptosis and/or by ADCC- and/or CDC-mediated mechanisms.

### Example 4

### Anti-Tumor Activity of Anti-CXCR4 in Combination with Anti-Pd-1 in CXCR4-Nonexpressing Mouse MC38 Tumor Model

The anti-tumor activity of different isotypes of the anti-mouse CXCR4 Ab was assessed, either alone or in combination with anti-mouse PD-1, in a CXCR4⁻ MC38 mouse colon adenocarcinoma model as described in Example 1. Anti-KLH in the mIgG1 and mIgG2a mAbs formats were used, singly or in combination, as non-binding control Abs.

The results for the controls Abs and single agents (anti-CXCR4 or anti-PD-1) are shown in FIG. 6A and the results for the combination treatments are shown in FIG. 6B. Whereas, like the Kp3 tumor model, MC38 cells do not express CXCR4 on the cell surface (see Example 1), MC38 tumors are fairly sensitive to anti-PD1 IgG1 treatment as disclosed in WO 2014/089113 and confirmed in FIG. 6A, unlike the Kp3 model (cf. FIG. 5A). A low level of single-agent activity was observed with CXCR4 IgG1 and CXCR4 IgG2a (FIG. 6A). In contrast, anti-PD-1 interacted synergistically with either anti-CXCR4 Ab isotype (IgG1 or IgG2a) to produce potent anti-tumor activity in this MC38 tumor model, with the anti-CXCR4 IgG2a combination being more efficacious than the anti-CXCR4 IgG1 combination (FIG. 6B). This result reinforces the indications from Example 3 that anti-CXCR4 may target CXCR4-expressing cells other than tumor cells including, for example, Tregs and/or MDSCs. The mIgG2a isotype of anti-CXCR4 may kill CXCR4⁺ Tregs and/or MDSCs by ADCC, ADCP and/or CDC in mice in addition to the mechanisms employed by the IgG1 isotype, including direct killing by apoptosis decreasing the trafficking of Tregs and/or MDSCs to the CXCL12-expressing tumor.

### Example 5

### Anti-Tumor Activity of Anti-CXCR4 IgG2A in Combination with Anti-PD-1 in CXCR4-Nonexpressing Mouse H22 Tumor Model

The anti-tumor activity of the anti-mouse CXCR4 mIgG2a Ab was assessed in combination with anti-mouse PD-1 mIgG1D265A in a CXCR4⁻ H22 mouse liver cancer model as described in Example 1. Anti-KLH in the mIgG1D265A and mIgG2a formats, corresponding to the isotypes of the anti-CXCR4 and anti-PD1 Abs used in the combination arms, were included as controls for isotype effects (secondary Fc-mediated interactions).

FIG. 7 shows tumor growth curves for individual mice treated with the combination of anti-PD-1 and anti-CXCR4 IgG2a (A), anti-PD-1 monotherapy (B) and the combination of anti-KLH isotype controls (C), and the median tumor growth curves are shown in FIG. 7D. Anti-PD-1 produced strong inhibition of tumor growth (FIG. 7B) compared to the controls which showed minimal inhibition of tumor growth (FIG. 7C), with three out of eight of the anti-PD-1-treated mice being tumor-free (TF) by Day 38. The combination with anti-mCXCR4 mIgG2a enhances the efficacy of anti-PD1 in the H22 model (FIG. 7A), with seven out of 8 mice TF by Day 31 for the combination versus three out of eight TF mice for PD-1 alone by Day 38 (FIG. 7B). This enhancement is clearly depicted in the median tumor growth curves shown in FIG. 7D. These data are consistent with the data obtained with the CXCR4⁻ Kp3 (Example 3) and MC38 (Example 4) tumors, substantiating the evidence that anti-CXCR4 can synergize with anti-PD-1 in augmenting the inhibition of tumor growth even of tumors that do not express CXCR4, probably by causing direct apoptosis or depletion of immunosuppressive MDSCs and/or Tregs.

### Example 6

### Design of Phase 1/2 Clinical Study of Ulocuplumab Combined with Nivolumab to Treat SCLC and PAC

### Study Design and Duration

This is an open-label, multicenter Phase 1/2 study of ulocuplumab in combination with nivolumab designed to independently evaluate the safety and efficacy in subjects with SCLC and PAC. The study design consists of a Dose Evaluation Phase (Stage 1) that includes a DLT evaluation for the dose levels of 400, 800 mg and 1600 mg weekly followed by a parallel evaluation of three cohorts to assess two dose levels (800 mg and 1600 mg weekly) and an additional schedule for 1600 mg (every 2 weeks). If 2 or more DLT are seen with any dose during the DLT evaluation period, a lower dose is evaluated as a single arm. A recommended dose is selected based on the safety and efficacy data from Stage 1 and proceeds to Dose Expansion in the form of a Simon optimal 2-stage-like design or a randomized Phase 2 study with comparative arm if high efficacy is observed (Simon, 1989).

The study consists of Screening, Treatment, and Follow-up. All subjects undergo a screening period to determine eligibility within 28 days prior to initial dosing. During the treatment phase, ulocuplumab is administered weekly or every two weeks (1600 mg dose only) and nivolumab is administered every two weeks. The treatment period continues until disease progression or occurrence of unacceptable toxicity. During follow-up, subjects are monitored for disease activity and safety. The duration of the study is anticipated to be approximately 2 years.

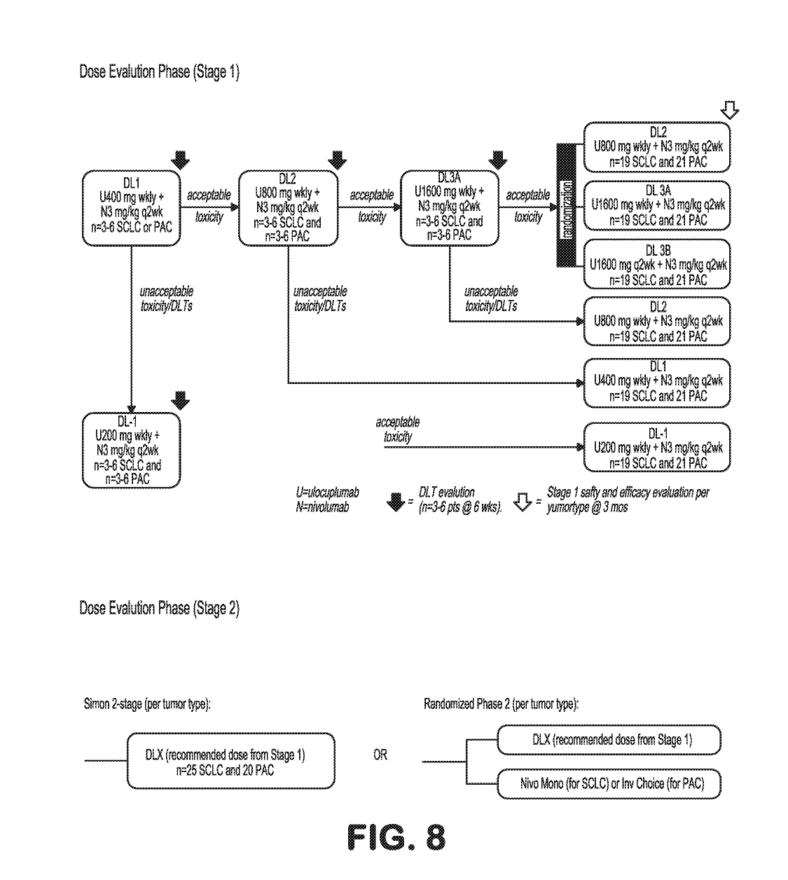

The study design schematic is presented in FIG. 8.

### Dose Evaluation Phase (Stage 1)

The Dose Evaluation Phase consists of a DLT evaluation period followed by an evaluation of up to three cohorts with various doses and schedules of ulocuplumab combined with nivolumab (see Table 1). The DLT evaluation period is conducted in the first 3-6 subjects with either PAC or SCLC at dose level 1 (DL1; 400 mg weekly of ulocuplumab combined with nivolumab), followed by 3-6 subjects each with PAC and SCLC at DL2 (800 mg weekly ulocuplumab combined with nivolumab), followed by 3-6 subjects each with PAC and SCLC at DL3A (1600 mg weekly ulocuplumab combined with nivolumab) for 6 weeks. For DL1, both tumor types are combined for the safety evaluation. For DL2 and DL3A, each tumor type is evaluated for safety independently in the event that tumor specific AEs emerge. Enrollment during the DLT evaluation phase allow for concurrent accrual of up to 6 subjects in each dose/tumor cohort (i.e., Rolling Six design) (Skolnik et al., 2007). This design allows for 3-6 evaluable subjects to contribute to the DLT evaluation depending upon how many are enrolled and still being evaluated during the DLT period. Decisions as to whether to enroll a new participant onto the current dose level or next highest dose level are based on available data at the time of new participant enrollment. Study stopping rules for the DLT evaluation period and the decision to proceed with the Dose Evaluation Phase include the following:
Enrollment in the active cohort proceeds if there are: fewer than 3 subjects enrolled, up to a maximum of 6 subjects; and 1 DLT in 2 or up to 5 subjects evaluable for toxicity.
Enrollment in the active cohort is paused if there are: a maximum of 6 subjects enrolled (including evaluable and non-evaluable).
Active cohort is deemed intolerable and enrollment will be permanently stopped if there are: 2 or more DLTs in up to 6 subjects evaluable for toxicity.
Active cohort is deemed tolerable and enrollment proceeds to next step if there are: 0 DLT in 3 or up to 6 subjects evaluable for toxicity; and 1 DLT in 6 subjects evaluable for toxicity.
Subjects who are not evaluable for DLT (i.e., discontinuation due to disease progression) are replaced with a concurrently enrolled subject.

TABLE 1
Dose levels for ulocuplumab and nivolumab

| Dose Level | Ulocuplumab | Nivolumab |
|---|---|---|
| DL-1 | 200 mg weekly | 3 mg/kg every 2 weeks |
| DL1 | 400 mg weekly | 3 mg/kg every 2 weeks |
| DL2 | 800 mg weekly | 3 mg/kg every 2 weeks |
| DL3A | 1600 mg weekly | 3 mg/kg every 2 weeks |
| DL3B | 1600 mg every 2 weeks | 3 mg/kg every 2 weeks |

Depending on the number of DLTs observed during the DLT evaluation period, escalation or de-escalation of ulocuplumab may be warranted. Dose escalation/de-escalation at the 800 mg weekly and 1600 mg weekly ulocuplumab dose levels occurs independently for each tumor type. No dose modification of nivolumab is allowed in this study.

If the toxicity at DL1 and DL2 and DL3A is acceptable, enrollment proceeds with three randomized cohorts (DL2, DL3A, and DL3B) to complete Stage 1.

If the toxicity at DL3A is unacceptable, enrollment proceeds at DL2 to complete Stage 1.

If the toxicity at DL2 is unacceptable, enrollment proceeds at DL1 to complete Stage 1.

If the toxicity at DL1 is unacceptable, a new DLT evaluation period is initiated at DL-1.

If the toxicity of DL-1 is unacceptable, enrollment is stopped for that tumor type.

If the toxicity at DL-1 is acceptable, enrollment proceeds at DL-1 to complete Stage 1 at a single dose level.

Decision Rules to Proceed with Dose Expansion Phase

An interim analysis (IA) is carried out when all subjects in the Dose Evaluation Phase in an individual tumor type have at least three months of treatment, or are discontinued prematurely. This IA is conducted independently for each tumor type. Investigator-assessed objective response rate (ORR) is used to guide the decision making for the Stage 2 portion of the study. However, all available efficacy and safety data are used to select the recommended dose that is further evaluated in the Dose Expansion Phase. Furthermore, if the level of efficacy observed at the recommended dose in the Dose Evaluation Phase does not warrant stopping evaluation of that tumor type, it is used to select the appropriate Expansion Phase study design, either proceeding with a Simon 2-stage-like design or conducting a randomized Phase 2 study with comparative arm. The efficacy thresholds (see Table 2) used for the IA analysis are based on the preliminary efficacy data from the ongoing Phase 1/2 study evaluating nivolumab monotherapy in SCLC and PAC and the level of activity reported for 2L options (NCT01928394; Hurwitz et al., 2015). The determination of low, moderate or high efficacy is based primarily on the response rates observed with ulocuplumab and nivolumab, but the totality of available safety and efficacy data is considered.

If the number of responders per tumor type at the recommended dose level is consistent with low efficacy, the evaluation of that tumor type is placed on hold pending final review of the data.

If the number of responders per tumor type at the recommended dose level is consistent with moderate efficacy, the Dose Expansion Phase continues with a single-arm evaluation.

If the number of responders per tumor type at the recommended dose level is consistent with high efficacy, the Dose Expansion Phase continues with a randomized Phase 2 study with comparative arm.

TABLE 2
Stage 1 efficacy threshold for each tumor type

| Efficacy | | |
|---|---|---|
| Threshold | SCLC | PAC |
| Low | ≤3 responders/19 subjects | ≤1 responders/21 subjects |
| Moderate | 4-8 responders/19 subjects | 2-5 responders/21 subjects |
| High | ≥9 responders/19 subjects | ≥6 responders/21 subjects |

### Dose Expansion Phase (Stage 2)

Based on the results of the IA, the Dose Expansion Phase consists of a second stage of a Simon 2-stage like single arm study (moderate efficacy) or a randomized Phase 2 study with comparative arm (high efficacy).

The second stage of a Simon 2-stage like design expands enrollment at the recommended dose level in a single arm study. An additional 25 SCLC subjects and 20 PAC subjects are enrolled to complete this evaluation. The primary endpoint is investigator-assessed ORR for both tumor types, and PFS is considered a secondary endpoint.

The randomized Phase 2 study compares the combination therapy at the recommended dose level versus a comparative arm appropriate for that tumor type. The primary endpoint of this study is dictated by the tumor type, where ORR is the endpoint for a randomized Phase 2 study in SCLC and overall survival (OS) for a randomized Phase 2 study in PAC. For ORR, an independent radiology review committee (IRRC) performs blinded independent review of the imaging per Response Evaluation Criteria in Solid Tumors (RECIST 1.1) criteria.

SCLC

A randomized Phase 2 study with comparative arm in SCLC compares the recommended dose of ulocuplumab combined with nivolumab versus nivolumab monotherapy. The main goal of this comparison is to determine whether the combination therapy is superior to nivolumab monotherapy. The primary endpoint of this study is evaluation of IRRC-assessed ORR. Safety, tolerability and PFS are considered as secondary endpoints. A randomized Phase 2 study requires an additional 50 subjects per arm (i.e., 100 for the two arms). The SCLC subjects included in the Dose Evaluation Phase are not part of the efficacy analysis of the randomized Phase 2 study. A stratification factor is used for this portion of the study to balance recruitment and includes performance status (ECOG 0 vs. 1).

PAC

A randomized Phase 2 study with comparative arm in PAC compares the recommended dose of ulocuplumab combined with nivolumab versus investigator's choice 2L chemotherapy. The main goal of this comparison is to determine if ulocuplumab plus nivolumab combination therapy is superior to 2L chemotherapy. The primary endpoint of this study is OS. Safety, tolerability and PFS are considered as secondary endpoints. For PAC, a randomized Phase 2 study requires an additional 125 subjects per arm (i.e., 250 for the two arms). IRRC-assessed ORR is considered an exploratory endpoint. The PAC subjects included in the Dose Evaluation Phase are not considered in the analysis of the randomized Phase 2 study. Investigator's choice chemotherapy options in this study are based on NCCN guidelines for PAC and include the following (NCCN GUIDELINES®, Version 2.2015—Pancreatic Adenocarcinoma; Tempero et al., 2012):

Subjects that fail FOLFIRINOX or other fluoropyrimidine-based regimens can consider gemcitabine-based therapies for this study; and

Subjects that fail gemcitabine-based regimens can consider fluoropyrimidine-based regimens for this study.

Stratification factors are used for this portion of the study and include performance status (ECOG 0 vs. 1) and type of chemotherapy used in the 1L setting (fluoropyrimidine-containing vs. gemcitabine-containing regimens).

### Dose Limiting Toxicity

The incidence of DLT(s) assessed in the first 3-6 evaluable subjects per tumor type (if applicable) during the first 6 weeks is used to initially determine whether a dose level is tolerable. A subject is considered evaluable for DLT if they receive at least 5 out of 6 ulocuplumab doses and at least 2 out of 3 nivolumab doses in a 6-week dosing period or experience a DLT. DLT is not an AE considered by the investigator to be disease related. The following drug-related AE (whether related to one or both agents) is considered a DLT:
Any drug-related non-hematological AE of Grade >3, including laboratory abnormalities. If a subject has baseline AST or ALT within the Grade 2 toxicity range, a DLT is considered for drug-related elevations in AST and/or ALT>2× baseline or >8×ULN;
Any drug-related hematological AE of Grade>4;
Any toxicity managed by discontinuation of ulocuplumab;
Any toxicity managed by discontinuation of nivolumab.

During the DLT period, subject withdrawal is required for any ulocuplumab dosing delay of more than 14 days.

### Treatment Beyond Disease Progression

Accumulating evidence indicates that subjects treated with immunotherapy may derive clinical benefit despite evidence of progressive disease (PD). Accordingly, subjects are permitted to continue with treatment beyond initial RECIST 1.1-defined PD as long as they show investigator-assessed clinical benefit and the subject is tolerating the study drugs. The assessment of clinical benefit takes into account whether the subject is clinically deteriorating and unlikely to receive further benefit from continued treatment.

Subjects discontinue study therapy upon evidence of further progression, defined as an additional 10% or greater increase in tumor burden from time of initial progression (including all target lesions and new measurable lesions). New lesions are considered measurable at the time of initial progression if the longest diameter is at least 10 mm (except for pathological lymph nodes, which must have a short axis of at least 15 mm). Any new lesion considered non-measurable at the time of initial progression may become measurable and therefore included in the tumor burden measurement if the longest diameter increases to at least 10 mm (except for pathological lymph nodes, which must have an increase in short axis to at least 15 mm).

For statistical analyses that include the investigator-assessed progression date, subjects who continue treatment beyond initial investigator-assessed, RECIST 1.1-defined progression are considered to have investigator-assessed progressive disease at the time of the initial progression event. Subjects who have tumor shrinkage following RECIST 1.1-defined progression are also descriptively summarized separately since these immune responses may be used in decision rules for selecting Dose Expansion Phase (Stage 2) study design.

### Example 7

### Efficacy Assessments

Baseline tumor assessments are performed within 28 days prior to the first dose utilizing contrast-enhanced Computed Tomography (CT) or magnetic resonance imaging (MRI) scans. In addition to chest, abdomen, pelvis, and brain, all known sites of disease are assessed at baseline. Subsequent assessments include chest, abdomen, and pelvis, and all known sites of disease and use the same imaging method as was used at baseline. Subjects are evaluated for tumor response beginning 6 weeks (±1 week) from first dose and continuing every 6 weeks (±1 week) for the first 24 weeks and every 12 weeks (±1 week) thereafter, until disease progression is documented or treatment is discontinued (whichever occurs later). Tumor assessments for ongoing study treatment decisions are completed by the investigator using RECIST 1.1 criteria.

### Primary Efficacy Assessment

The primary efficacy endpoint is ORR, as determined by the investigators, for the Dose Evaluation Phase and if a Simon 2-stage like design is selected for the Expansion Phase. If an open label randomized Phase 2 with a comparative arm is selected for the Expansion Phase, the primary efficacy endpoint is ORR for SCLC and OS for PAC. If a randomized Phase 2 study is initiated for either tumor type, a blinded independent review of all imaging scans is used to determine ORR, best overall response (BOR) and the magnitude of reduction in tumor volume. For OS, every effort is made to collect survival date on all randomized subjects (including subjects who withdraw from treatment for any reason) who are eligible to participate in the study and who have not withdrawn consent for survival data collection. If the death of a subject is not reported, every date collected in this study representing a date of subject contact is used in determining the subject's last known alive date.

### Endpoints

Primary Endpoint(s)

The incidence of DLTs is the primary safety endpoint during the DLT evaluation phase.

In terms of efficacy, the primary endpoint for SCLC is investigator-assessed ORR for the Dose Evaluation Phase and the single arm Dose Expansion Phase. If the randomized Phase 2 study in SCLC subjects is triggered, an IRRC performs blinded independent review of the imaging per RECIST 1.1 criteria for the assessment of ORR. The ORR is defined as the number of subjects with a best overall response (BOR) of complete response (CR) or partial response (PR) divided by the number of treated subjects (the number of randomized subjects for the randomized Phase 2 study with comparative arm). The BOR is defined as the best response designation, as determined by the investigator, recorded between the first dosing date (randomization date for the randomized Phase 2 study with comparative arm) and the date of objectively documented progression per RECIST 1.1 or the date of subsequent anti-cancer therapy, whichever occurs first. CR or PR determinations included in the BOR assessment are confirmed by a second scan no less than 4 weeks after the criteria for response are first met. For subjects without documented progression or subsequent therapy, all available response designations contribute to the BOR assessment. For subjects who continue treatment beyond progression, the BOR is determined based on response designations recorded up to the time of the initial RECIST 1.1-defined progression.

For PAC, the primary endpoint is investigator assessed ORR for the Dose Evaluation Phase and the single arm Dose Expansion Phase. OS is the primary endpoint for the randomized two-arm Phase 2 study. The ORR is defined as above, and OS is defined as the time between the randomization date and the date of death due to any cause. A subject who has not died is censored at the last known alive date.

Secondary Endpoint(s)

Safety and tolerability are analyzed through the incidence of DLTs, adverse events, serious adverse events, and specific laboratory abnormalities (worst grade). Toxicities are graded using the NCI CTCAE version 4.0.

PFS is defined as the time from first dosing date (randomization date for the randomized Phase 2 study with comparative arm) to the date of the first documented tumor progression, as determined by the investigator (per RECIST 1.1), or death due to any cause, whichever occurs first. Subjects who die without a reported prior progression are considered to have progressed on the date of their death. Subjects who did not progress or die are censored on the date of their last evaluable tumor assessment. Subjects who did not have any on-study tumor assessments and did not die are censored on the date of their first dosing date (randomization date for the randomized Phase 2 study with comparative arm). Subjects who started anti-cancer therapy without a prior reported progression are censored on the date of their last evaluable tumor assessment prior to the initiation of subsequent anti-cancer therapy.

Exploratory Endpoint(s)

Duration of response (DOR) is computed for subjects with a BOR of PR or CR and is defined as the time from when measurement criteria are first met for CR or PR (whichever status is recorded first) to the date of the first documented tumor progression as determined using RECIST 1.1 criteria or death due to any cause, whichever occurs first. For subjects who neither progress nor die, the DOR is censored on the date of their last evaluable tumor assessment.

Disease control rate is defined as the ORR above except that its definition includes a BOR of PR or CR or stable disease (for at least 6 weeks, present on 2 consecutive scans, the second scan a minimum of 10 weeks from baseline) divided by the number of treated subjects (the number of randomized subjects if a randomized Phase 2 study with comparative arm is initiated).

OS as an exploratory endpoint is defined as for the primary endpoint, considering the randomization date for the randomized Phase 2 study with comparative arm and the dose start date for the other designs.

ORR is further characterized by the magnitude of reduction in tumor volume. The magnitude of reduction in tumor volume is defined as the percent decrease in tumor volume from baseline to nadir, observed up until the time of the first documented tumor progression or death. As an exploratory objective for the randomized Phase 2 study in PAC subjects, IRRC-assessed ORR is used.

### Analyses

All analyses are presented separately by tumor type.

Efficacy Analyses

For the Dose Evaluation Phase, efficacy analyses are summarized using the All Dose-evaluation Randomized Treated Subjects by randomized cohort (primary population). Additionally, analyses including all efficacy data collected during that Phase using the All Treated Subjects population are provided by regimen. Analyses are presented as-treated.

If the form of the Expansion Phase is a Simon 2-stage like design, efficacy analyses are summarized for the regimen recommended for the Dose Expansion Phase, pooling data from the related randomized cohort from the All Dose-evaluation Randomized Treated Subjects during Stage 1 with the Stage 2 data (primary population). Additionally, analyses using the All Treated Subjects and including all efficacy data collected for that regimen during the Dose Evaluation and the Expansion Phases are provided. Analyses are presented as-treated.

If a randomized Phase 2 study with comparative arm is initiated, efficacy analyses are presented separately by treatment arm using the All Expansion Phase Randomized Subjects. Analyses are presented as-randomized.

Primary Endpoint Methods

ORR is summarized by a binomial response rate and corresponding two-sided 90% exact CI using the Clopper and Pearson method. If a randomized Phase 2 study with comparative arm is initiated, ORR is compared between the treatment arms using a one-sided alpha level of 0.10 with Cochran-Mantel-Haenszel (CMH) test stratified by the stratification factors defined for each tumor type. A two-sided, 80% CI for the difference in response rates is also computed, adjusting for the stratification factors.

The primary analysis of OS as primary endpoint for PAC is a comparison of the OS of subjects randomized to ulocuplumab plus nivolumab to that of subjects randomized to the investigator's choice chemotherapy using a one-sided alpha level of 0.10 log-rank test stratified by the stratification factors defined for each tumor type. The hazard ratio and associated two-sided 80% confidence interval are computed using an univariate Cox proportional hazards model with treatment as the sole covariate. Further analyses of OS are summarized descriptively using Kaplan-Meier methodology. Median values of OS, along with two-sided 95% CIs using the Brookmeyer and Crowley method considering a log-log transformation, are calculated. OS rates at 3, 6, 9, 12, 18 and 24 months are estimated as well as associated two-sided 95% CIs considering a log-log transformation.

Secondary Endpoint Methods

PFS as a secondary endpoint is descriptively summarized as for OS. PFS rates at 3, 6, 9, 12, and 18 months are estimated as well as associated two-sided 95% CIs considering a log-log transformation.

Exploratory Endpoint Methods

ORR as exploratory endpoint is descriptively summarized as for the primary endpoint. DOR is summarized for subjects who achieve confirmed PR or CR using the Kaplan-Meier (KM) product-limit method. The median value along with two-sided 95% CI using the Brookmeyer and Crowley method considering a log-log transformation is also calculated. In addition, the percentage of responders still in response at different time points (3, 6, 12, and 18 months) is presented based on the KM plot.

The magnitude of reduction in tumor burden is summarized descriptively.

Disease control rate is summarized by a binomial response rate and corresponding two-sided 95% exact CI using the Clopper and Pearson method.

OS as an exploratory endpoint for SCLC subjects is descriptively summarized as for the primary endpoint for PAC subjects.

Safety Analyses

Except where indicated, safety analyses are performed using the All Treated Subjects population and are presented as-treated.

During the DLT evaluation Phase, the primary analysis consists of the incidence of DLTs among DLT-evaluable Subjects but all available safety and tolerability data are used to assess the safety of the regimens.

For the Dose Evaluation Phase, safety analyses are summarized by randomized cohort. Additionally, analyses including all safety data collected during that Phase are provided by regimen.

If the form of the Expansion Phase is a Simon 2-stage like design, safety analyses are summarized for the regimen recommended for the Dose Expansion, pooling data from both stages of the study. Additionally, analyses including all safety data collected for that regimen during the Dose Evaluation and the Expansion Phases are provided.

If a randomized Phase 2 study with comparative arm is initiated, safety analyses are presented separately by treatment arm.

Events (AEs or laboratory) are counted as on-study if the event occurred within 100 days of the last dose of ulocuplumab or within 100 days of the last dose of nivolumab, whichever is later. All on-study AEs, treatment-related AEs, SAEs, treatment-related SAEs, AEs leading to discontinuation and treatment-related AEs leading to discontinuation are tabulated (All Grades and Grade 3-4) using worst grade per NCI CTCAE v 4.0 criteria by system organ class and preferred term. On-study laboratory abnormalities including hematology, chemistry, liver function, and renal function are summarized (All Grades and Grade 3-4) using worst grade NCI CTCAE v 4.0 criteria.

### Interim Analyses

Within each tumor type, an interim analysis (IA) is conducted when all subjects in the Dose Evaluation Phase have a minimum of 3 months of treatment or discontinued prematurely. The objectives of this IA are: (1) to determine if further study of ulocuplumab combined with nivolumab is warranted in the tumor type; (2) if further study is warranted, to select a recommended dose for the Dose Expansion Phase; and (3) if the Dose Expansion Phase is to be completed, to determine whether to conduct a single arm second stage of a Simon optimal 2-stage like design or an open label randomized Phase 2 design with a comparative arm.

The decision to further study ulocuplumab combined with nivolumab in each tumor type is primarily based on the pre-defined Simon 2-stage design thresholds for the Dose Evaluation Phase (at least 4 responders for SCLC and at least 2 responders for PAC). In addition, the selection of the recommended dose is based on all available safety and efficacy data for that IA from both tumor types. The decision to proceed from the Dose Evaluation Phase to the Dose Expansion Phase is conducted for each tumor type independently. Consideration may be given to evaluating final data before a decision is reached to stop further study of the combination to ensure that the full characterization of the response pattern is evaluated.

The decision to proceed with an open label randomized two-arm Phase 2 design rather than completing the second stage of a Simon optimal 2-stage like design for the Expansion Phase is taken if, among the treated subjects in the recommended dose selected during the Dose Evaluation Phase, a “high” frequency of responders is observed. For SCLC, this “high” number is at least 9 responders and, for PAC, at least 6 responders.

This number of responders has been defined considering clinical input but, ensuring that the related proportion of responders also presents with a 90% exact CI lower limit above 25% for SCLC or above 12% for PAC. These percentages correspond to the minimum proportion of responders that would be needed at the end of a Simon 2-stage design in order to further evaluate the drug (for SCLC, 11 responders among the 44 subjects is 25% and, for PAC, 5 responders among the 41 subjects is 12%).

During the Expansion phase, an IA is conducted when all subjects of the second stage of the Simon 2-stage like design have a minimum of 3 months of treatment or discontinued prematurely. If a randomized Phase 2 study with comparative arm is initiated, IA is conducted for the DMC as specified in the DMC charter on a regular basis.

### Example 8

### Pharma Cokinetic and Immunogenicity Assessments

A detailed schedule of PK and immunogenicity evaluations is provided in Table 3 and Table 4. Pre-dose samples are taken within 30 minutes prior to the start of the first infusion for the day. End of infusion samples are taken just prior to the end of infusion, preferably within 2 min, of the respective study drug. All other time points are relative to the start of infusion for the respective study drug. All on-treatment PK time points are intended to align with days on which study drug is administered; if dosing occurs on a different day due to minor scheduling shifts, the PK sampling is adjusted accordingly.

For a randomized Phase 2 study with comparative arm in SCLC, nivolumab PK and immunogenicity sample collection follow Table 4 for the nivolumab monotherapy comparator arm. For a randomized Phase 2 study with comparative arm in PAC, no PK and immunogenicity samples are collected for the comparator arm with the Investigator's Choice 2L chemotherapy.

TABLE 3
Pharmacokinetic and immunogenicity sampling schedule for ulocuplumab and
nivolumab in Dose Evaluation Phase (Stage 1)

| | | Time | Time | | | | |
|---|---|---|---|---|---|---|---|
| | | hour:min | hour:min | | | | |
| | | (Relative to | (Relative to | | | | |
| | | start of | start of | | | Immunogenicity | |
| Sample collection Time | | ulocuplumab | nivolumab | PK Sample | | Sample^{a} | |
| Study Day | Time (Event) | infusion) | infusion) | Ulocuplumab | Nivolumab | Ulocuplumab | Nivolumab |
| Day 1, Week 1 | 0 h (Pre-dose) | 00:00 | 00:00 | X | X | X | X |
| | 1 h (EOI | 01:00 | | X | | | |
| | Ulocuplumab)^{b} | | | | | | |
| | 2.5 h (EOI | 02:30 | 01:00 | X | X | | |
| | Nivolumab)^{c} | | | | | | |
| | 4 h | 04:00 | | X | | | |
| | 6 h | 06:00 | | X | | | |
| Day 2, Week 1 | 24 h | 24:00 | | X | | | |
| Day 3, Week 1 | 48 h | 48:00 | | X | | | |
| Day 4, 5 or 6, | 72-120 h | 72:00-120:00 | | X | | | |
| Week 1 | | | | | | | |
| Day 1, Week 2 | 0 h (Pre-dose)/ | 00:00/ | | X | | | |
| | 168 h^{d} | 168:00^{c} | | | | | |
| Day 1, Week 3, | 0 h (Pre-dose) | 00:00 | 00:00 | X | X | X | X |
| 5, 7, 13, 19, 25 | 1 h (EOI | 01:00 | | X | | | |
| | Ulocuplumab)^{b} | | | | | | |
| | 2.5 h (EOI | 02:30 | 01:00 | X | X | | |
| | Nivolumab)^{c} | | | | | | |
| Day 1 of every | 0 h (Pre-dose) | 00:00 | 00:00 | X | X | X | X |
| 12th week | 1 h (EOI | 01:00 | | X | | | |
| (starting from | Ulocuplumab)^{b} | | | | | | |
| Week 37) | 2.5 h (EOI | 02:30 | 01:00 | X | X | | |
| | Nivolumab)^{c} | | | | | | |
| End of | | | | X | X | X | X |
| Treatment/ | | | | | | | |
| Discontinuation | | | | | | | |
| Follow-up^{e} | | | | X | X | X | X |

^{a}Serum sample for immunogenicity assessment is collected within 30 min before start of the first infusion of the day.
^{b}End of infusion (ulocuplumab): This sample is taken immediately prior to stopping the ulocuplumab infusion (preferably within 2 min prior to end of infusion). If the end of ulocuplumab infusion is delayed, the collection of the infusion is delayed accordingly.
^{c}End of infusion (nivolumab): This sample is taken immediately prior to stopping the nivolumab infusion (preferably within 2 min prior to end of infusion). The 2.5-h time point takes into account 30 min in between ulocuplumab and nivolumab dosing. If the end of nivolumab infusion is delayed, the collection of this sample is delayed accordingly.
^{d}For ulocuplumab weekly dosing, a pre-dose sample (relative time is 00:00) is collected; for ulocuplumab given every 2 weeks, a 168-h sample (relative time is 168:00) is collected.
^{e}First 2 follow-up visits (up to 100 days from end of treatment visit except for subjects that withdraw consent).

TABLE 4
Pharmacokinetic and immunogenicity sampling schedule for ulocuplumab and
nivolumab in Dose Expansion Phase (Stage 2)

| | | Time | Time | | | | |
|---|---|---|---|---|---|---|---|
| | | hour:min | hour:min | | | | |
| | | (Relative to | (Relative to | | | | |
| | | start of | start of | | | Immunogenicity | |
| Sample collection Time | | ulocuplumab | nivolumab | PK Sample | | Sample^{a} | |
| Study Day | Time (Event) | infusion) | infusion) | Ulocuplumab | Nivolumab | Ulocuplumab | Nivolumab |
| Day 1 of Weeks | 0 h (Pre-dose) | 00:00 | 00:00 | X | X | X | X |
| 1, 3, 5, 7, 13, | 1 h (EOI | 01:00 | | X | | | |
| 19, 25 | Ulocuplumab)^{b} | | | | | | |
| | 2.5 h (EOI | 02:30 | 01:00 | | X | | |
| | Nivolumab)^{c} | | | | | | |
| Day 1 of 12th | 0 h (Pre-dose) | 00:00 | 00:00 | X | X | X | X |
| (week starting | 1 h (EOI | 01:00 | | X | | | |
| from Week 37) | Ulocuplumab)^{b} | | | | | | |
| | 2.5 h (EOI | 02:30 | 01:00 | | X | | |
| | Nivolumab)^{c} | | | | | | |
| End of | | | | X | X | X | X |
| Treatment/ | | | | | | | |
| Discontinuation | | | | | | | |
| Follow-up^{d} | | | | X | X | X | X |

^{a}Serum sample for immunogenicity assessment is collected within 30 min before start of the first infusion of the day.
^{b}End of infusion (ulocuplumab): This sample is taken immediately prior to stopping the ulocuplumab infusion (preferably within 2 min prior to end of infusion). If the end of ulocuplumab infusion is delayed, the collection of the infusion is delayed accordingly.
^{c}End of infusion (nivolumab): This sample is taken immediately prior to stopping the nivolumab infusion (preferably within 2 min prior to end of infusion). The 2.5-h time point takes into account 30 min in between ulocuplumab and nivolumab dosing. If the end of nivolumab infusion is delayed, the collection of this sample is delayed accordingly
^{d}Follow-up visits (up to 100 days from end of treatment visit except for subjects that withdraw consent).

Pharmacokinetic Analyses

The ulocuplumab and nivolumab concentration data obtained in this study may be combined with data from other studies in the clinical development program to develop or refine a population PK model. This model is used to evaluate the effects of intrinsic and extrinsic covariates on the PK of ulocuplumab and nivolumab and to determine measures of individual exposure (such as steady-state peak, trough, and time-averaged concentration). In addition, model determined exposures may be used for exposure-response analyses. Results of population PK and exposure response-analyses are reported separately.

### Example 9

### Biomarker Assessments

Peripheral blood and tumor tissue are collected prior to therapy and at selected time points on treatment. Biomarker sampling schedules are provided in Table 5 and Table 6.

Soluble Biomarkers

Inflammatory cytokines, chemokines and other exploratory serum-based biomarkers are characterized and quantified prior to treatment and at selected time points post-treatment as potential PD markers. Two cancer-related biomarkers, C-Reactive Protein (CRP) and cancer antigen 19.9 (CA19.9), are evaluated prior to treatment and at selected time points post-treatment as potential markers of disease activity.

Immunophenotyping

The proportion of specific lymphocyte subsets and expression levels of T cell co-stimulatory markers in peripheral blood mononuclear cell (PBMC) preparations is quantified by flow cytometry. Analyses may include, but not necessarily be limited to, the proportion of T, B, and NK cells, proportion of myeloid-derived suppressor cells (MDSCs), proportion of memory and effector T cell subsets, and expression levels of PD-1, PD-L1, ICOS, and Ki67.

TABLE 5
Biomarker sampling schedule for dose evaluation phase (Stage 1)

| | | | Whole Blood | |
|---|---|---|---|---|
| | | | for Receptor | Whole Blood |
| Sample Collection Time | | Tumor | Occupancy and | for CD34⁺ |
| Study Day | Time (Event) | Biopsy | T cell counts | Cell Counts |
| Screening | | X | | |
| Day 1, Week 1 | 0 h (pre-dose) | | X | X |
| | 4.0 h | | X | X |
| Day 2, Week 1 | 24 h | | X | X |
| Day 3, Week 1 | 48 h | | X | X |
| Day 4, 5 or 6, | 72-120 h | | X | X |
| Week 1 | | | | |
| Day 1, Week 2 | 0 h (pre-dose)/ | | X | X |
| | 168 h^{a} | | | |
| Day 1, Week 3, | 0 h (pre-dose) | | X | X |
| 5, 7, 13, 19, 25 | 1 h (EOI) | | X | X |

^{a}For ulocuplumab weekly dosing, collect a pre-dose sample (relative time is 00:00); for ulocuplumab given every 2 weeks, collect a 168 hour sample (relative time is 168:00).

TABLE 6
Biomarker sampling schedule for dose expansion phase (Stage 2)

| | | | | Whole | |
|---|---|---|---|---|---|
| | | | Whole | Blood | |
| Sample collection Time | | Tumor | Blood | for PBMC | Serum |
| Study Day | Time (Event) | Biopsy | for RNA | isolation | Analysis |
| Screening | | X | | | |
| Day 1, | 0 h (pre-dose) | | X | X | X |
| Week 1 | | | | | |
| Day 1, | 0 h (pre-dose) | | X | X | X |
| Week 13 | | | | | |
| Day 1, | 0 h (pre-dose) | | X | X | X |
| Week 25 | | | | | |

Peripheral Blood Gene Expression

The expression level of genes related to response to nivolumab monotherapy and nivolumab/ulocuplumab combination therapy are quantified using whole blood samples. Analysis may include, but not necessarily be limited to, genes associated with immune-related pathways, such as T cell activation and antigen processing and presentation.

Receptor Occupancy Analysis

CXCR4 RO analysis is performed on circulating T cells as a surrogate biomarker of target binding by ulocuplumab. Data from these analyses is also used to facilitate interpretation of corresponding PK data. Absolute T cell and CD34⁺ cell counts are also assessed. Increases in absolute T cell and CD34⁺ cell counts post-dose are used together with the RO assay to confirm CXCR4 engagement and inhibition by ulocuplumab.

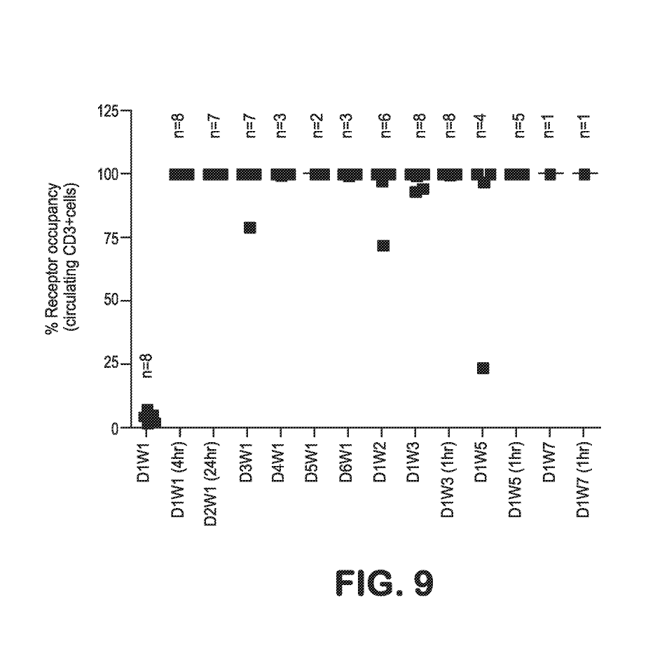

Preliminary RO data have been obtained in this ongoing clinical study for 8 subjects in the 200 mg ulocuplumab dose cohort. Within 4 h post dose with ulocuplumab, 100% RO (median value) was achieved and maintained at essentially all subsequent time points analyzed (FIG. 9). In one subject, % RO dropped to 23% at Day 1 of Week 5, but this was due to a delay of dosing due to a SAE unrelated to study drug at Day 1 of Week 4.

Tumor Biomarkers

Tumor biopsy specimens (fresh or archived material) are required from all subjects prior to treatment to characterize immune cell populations, expression of selected tumor markers, and for gene expression analysis. These samples are also used to assess expression and localization of CXCR4 and, if technically feasible, FAP and CXCL12, within the tumor and surrounding stroma. Biopsy samples are used for characterizing tumor infiltrating lymphocytes (TILs) and tumor antigens, analysis of T cell repertoire, and gene expression profiling.

Characterization of TILs and Tumor Antigens

Immunohistochemistry (IHC) is used to assess the number and composition of immune infiltrates in order to define the immune cell subsets present within tumor tissue before and after exposure to therapy. These IHC analyses may include, but not necessarily be limited to, the following markers: CD4, CD8, FOXP3, PD-1, PD-L1, and PD-L2.

T Cell Repertoire Analysis

In order to explore whether a diverse T cell repertoire is predictive of response to therapy, DNA isolated from tumor tissue is sequenced to quantify the composition of the T cell repertoire prior to, and during, monotherapy and combination therapy.

Gene expression profiling Tumor biopsies are examined for expression of selected immune related genes pre- and post-treatment.

Characterization of CXCL12, CXCR4 and FAP Expression

To ascertain whether CXCL12-mediated T cell inhibition is functional in solid tumors, the expression of CXCR4 and, if technically feasible, FAP and CXCL12 in tumor tissue, is assessed pre- and at post-treatment. Expression of CXCR4 and FAP is assessed by IHC, and CXCL12 expression is assessed via RNAscope.

### Sequence Listing Summary

| SEQ | |
|---|---|
| ID | |
| NO: | Description |
| 1 | V_{H} amino acid sequence of nivolumab (anti-PD-1) |
| 2 | V_{L} amino acid sequence of nivolumab (anti-PD-1) |
| 3 | Heavy chain amino acid sequence of nivolumab (anti-PD-1) |
| 4 | Light chain amino acid sequence of nivolumab (anti-PD-1) |
| 5 | V_{H} amino acid sequence of BMS-936559 (anti-PD-L1) |
| 6 | V_{L} amino acid sequence of BMS-936559 (anti-PD-L1) |
| 7 | Heavy chain amino acid sequence of BMS-936559 (anti-PD-L1) |
| 8 | Light chain amino acid sequence of BMS-936559 (anti-PD-L1) |
| 9 | V_{H} amino acid sequence of ulocuplumab (anti-CXCR4) |
| 10 | V_{L} amino acid sequence of ulocuplumab (anti-CXCR4) |
| 11 | Heavy chain amino acid sequence of ulocuplumab (anti-CXCR4) |
| 12 | Light chain amino acid sequence of ulocuplumab (anti-CXCR4) |
| 13 | Heavy chain amino acid sequence of IgG1f variant of ulocuplumab |
| | (anti-CXCR4) |
| 14 | Heavy chain amino acid sequence of IgG3b0 variant of |
| | ulocuplumab (anti-CXCR4) |
| 15 | V_{H} amino acid sequence of 2A5 (anti-CXCL12) |
| 16 | V_{L} amino acid sequence of 2A5 (anti-CXCL12) |
| 17 | Heavy chain amino acid sequence of 2A5 (anti-CXCL12) |
| 18 | Light chain amino acid sequence of 2A5 (anti-CXCL12) |

### REFERENCES

Ansell S M, Lesokhin A M, Borrello I, Halwani A, Scott E C (2015) PD-1 Blockade with nivolumab in relapsed or refractory Hodgkin's lymphoma. N Engl J Med 372:311-9.
Bai S, Jorga K, Xin Y, Jin D, Zheng Y et al. (2012) A guide to rational dosing of monoclonal antibodies. Clin Pharmacokinet 51(2): 119-35.
Balkwill F (2004) The significance of cancer cell expression of the chemokine receptor CXCR4. Semin Cancer Biol 14:171-9.
Becker P S, Foran J, Altman J, Yacoub A, Castro J et al. (2014) Targeting the CXCR4 pathway: Safety, tolerability and clinical activity of BMS-936564 (ulocuplumab), an anti-CXCR4 antibody, in relapsed refractory acute myeloid leukemia. 56th American Society of Hematology (ASH) Annual Meeting, San Francisco, Dec. 6-9, 2014, Oral Presentation No. 386.
Brahmer J R, Drake C G, Wollner I, Powderly J D, Picus J et al. (2010) Phase I study of single-agent anti-programmed death-1 (MDX-1106) in refractory solid tumors: safety, clinical activity, pharmacodynamics, and immunologic correlates. J Clin Oncol 28:3167-75.
Brahmer J R, Tykodi S S, Chow L Q, Hwu W J, Topalian S L et al. (2012) Safety and activity of anti-PD-L1 antibody in patients with advanced cancer. N Engl J Med 366:2455-65.
Burger J A, Kipps T J (2006) CXCR4: a key receptor in the crosstalk between tumor cells and their microenvironment. Blood 107:1761-7.
Burger M, Glodeck A, Hartmann T, Schmitt-Graff A, Silberstein L E et al. (2003) Functional expression of CXCR4 (CD184) on small-cell lung cancer cells mediates migration, integrin activation and adhesion to stromal cells. Oncogene 22: 8093-101.
Burger J A, Stewart D J, Wald O, Peled A (2011) Potential of CXCR4 antagonists for the treatment of metastatic lung cancer. Expert Rev Anticancer Ther 11(4):621-30.
Califano R, Abidin A Z, Peck R, Faivre-Finn C, Lorigan P (2012) Management of small cell lung cancer: Recent developments for optimal care. Drugs 72: 471-90.
Chen D S, Mellman I (2013) Oncology meets immunology: the cancer-immunity cycle. Immunity 39(1):1-10.
Chen Y, Ramjiawan R R, Reiberger T, Ng M R, Hato T et al. (2015) CXCR4 inhibition in tumor microenvironment facilitates anti-PD-1 immunotherapy in sorafenib-treated HCC in mice. Hepatology 61(5):1591-602.
Chute J P, Chen T, Feigal E, Simon R, Johnson B E (1999) Twenty years of phase III trials for patients with extensive-stage small-cell lung cancer: perceptible progress. J Clin Oncol 17:1794-801.
Conroy T, Desseigne F, Ychou M et al. (2011) FOLFIRINOX versus gemcitabine for metastatic pancreatic cancer. New Engl J Med 364(19):1817-25.
Domanska U M, Kruizinga R C, Nagengast W B, Timmer-Bosscha H et al. (2013) A review on CXCR4/CXCL12 axis in oncology: No place to hide. Eur J Cancer 49:219-30.
Duda D G, Kozin S V, Kirkpatrick N D, Xu L et al. (2011) CXCL12 (SDF-1)-CXCR4/CXCR7 pathway inhibition: An emerging sensitizer for anticancer therapies? Clin Cancer Res 17:2074-80.
Fearon D T (2014) The carcinoma-associated fibroblast expressing fibroblast activation protein and escape from immune surveillance. Cancer Immunol Res 2(3):187-93.
Feig C, Jones J O, Kraman M, Wells R J B, Deonarine A et al. (2013) Targeting CXCL12 from FAP-expressing carcinoma-associated fibroblasts synergizes with anti-PD-L1 immunotherapy in pancreatic cancer. Proc Natl Acad Sci USA 110 (50):20212-7.
Gangadhar T, Nandi S, Salgia R (2010) The role of chemokine receptor CXCR4 in lung cancer. Cancer Biol Ther 15:9(6):409-16.
Gao Z, Wang X, Wu K, Zhao Y, Hu G (2010) Pancreatic stellate cells increase the invasion of human pancreatic cancer cells through the stromal cell-derived factor-1/CXCR4 axis. Pancreatology 10(23):186-93.
Ghobrial I, Perez R, Baz R, Richardson P, Anderson K et al. (2014) Phase Ib study of the novel anti-CXCR4 antibody ulocuplumab (BMS-936564) in combination with lenalidomide plus low-dose dexamethasone, or with bortezomib plus dexamethasone in subjects with relapsed or refractory multiple myeloma. 56th American Society of Hematology (ASH) Annual Meeting, San Francisco, Dec. 6-9, 2014, Poster Presentation No. 3483.
Hamid O, Carvajal R D (2013) Anti-programmed death-1 and anti-programmed death-ligand 1 antibodies in cancer therapy. Expert Opin Biol Ther 13(6):847-61.
Hamid O, Robert C, Daud A, Hodi F S et al. (2013) Safety and tumor responses with lambrolizumab (anti-PD-1) in melanoma. New Engl J Med 369(2): 134-44.
Hartmann T N, Burger J A, Glodeck A, Fujii N, Burger M et al. (2005) CXCR4 chemokine receptor and integrin signaling co-operate in mediating adhesion and chemoresistance in small cell lung cancer (SCLC) cells. Oncogene 24:4462-71.
Herbst R S, Soria J C, Kowanetz M, Fine G D, et al. (2014) Predictive correlates of response to the anti-PD-L1 antibody MPDL3280A in cancer patients. Nature 515: 563-7.
Hodi F S, O'Day S J, McDermott D F, Weber R W et al. (2010) Improved survival with ipilimumab in patients with metastatic melanoma. N Engl J Med 363:711-23.
Hollinger P, Hudson P J (2005) Engineered antibody fragments and the rise of single domains. Nature Biotech 23(9): 1126-36.
Hurwitz H, Uppal N, Wagner S A, Bendell J C, Beck J T et al. (2015) A randomized double-blind phase 2 study of ruxolitinib (RUX) or placebo (PBO) with capecitabine (CAPE) as second-line therapy in patients with metastatic pancreatic cancer. American Society of Clinical Oncology (ASCO) oral presentation, Chicago, Ill., May 29-Jun. 2, 2015.
Janne P A, Freidlin B, Saxman S et al. (2002) Twenty-five years of clinical research for patients with limited-stage small cell lung carcinoma in North America. Cancer 95:1528-38.
Johnson D B, Wallender E K, Cohen D N, Likhari S S, Zwerner J P et al. (2013) Severe cutaneous and neurologic toxicity in melanoma patients during vemurafenib administration following anti-PD-1 therapy. Cancer Immunol Res 1:373-77.
Kabat E A, Wu T T, Perry H, Gottesman K, Foeller C et al. (1991) Sequences of Proteins of Immunological Interest, Fifth Edition, U.S. Department of Health and Human Services, NIH Publication No. 91-3242.
Kuhne M R, Mulvey T, Belanger B, Chen S et al. (2013) BMS-936564/MDX-1338: A fully human anti-CXCR4 antibody induces apoptosis in vitro and shows antitumor activity in vivo in hematologic malignancies. Clin Cancer Res 19:357-366.
Lesokhin A M, Callahan M K, Postow M A, Wolchok J D (2015) On being less tolerant: enhanced cancer immunosurveillance enabled by targeting checkpoints and agonists of T cell activation. Sci Transl Med 7(280):280sr1.
Lipson E J, Sharfman W H, Drake C G, Wollner I, Taube J M et al. (2013) Durable cancer regression off-treatment and effective reinduction therapy with an anti-PD-1 antibody. Clin Cancer Res 19:462-8.
McDermott D F, Atkins M B (2013) PD-1 as a potential target in cancer therapy. Cancer Med 2(5):662-73.
NCCN Guidelines Version 2.2015—Pancreatic Adenocarcinoma.
NCCN Guidelines Version 1.2016—Small Cell Lung Cancer.
NCCN GUIDELINES® (2015), available at: http://www.nccn.org/professionals/physician_gls/f_guidelines.asp#site, last accessed Jun. 8, 2015.
Nomi T, Sho M, Akahori T, Hamada K, Kubo A et al. (2007) Clinical significance and therapeutic potential of the Programmed Death-1 Ligand/Programmed Death-1 pathway in human pancreatic cancer. Clin Cancer Res 13(7):2151-7.
Olafsen T, Wu A M (2010) Antibody vectors for imaging. Semin Nucl Med 40(3):167-81.
Otani Y, Kijima T, Kohmo S, Oishi S, Minami T et al. (2012) Suppression of metastates of small cell lung cancer cells in mice by a peptidic CXCR4 inhibitor TF14016. FEBS Lett 586:3639-44.
Pardoll D M (2012) The blockade of immune checkpoints in cancer immunotherapy. Nat Rev Cancer 12:252-64.
Passaro D, Irigoyen M, Catherinet C, Gachet S, Da Costa De Jesus C et al. (2015) CXCR4 Is Required for Leukemia-Initiating Cell Activity in T Cell Acute Lymphoblastic Leukemia. Cancer Cell 27(6):769-79.
PCT Publication No. WO 2008/060367, published May 22, 2008 by Medarex, Inc.
PCT Publication No. WO 2008/142303, published Nov. 27, 2008 by Pierre Fabre Medicament.
PCT Publication No. WO 2009/140124, published Nov. 19, 2009 by Eli Lilly and Co.
PCT Publication No. WO 2010/037831, published Apr. 8, 2010 by Pierre Fabre Medicament.
PCT Publication No. WO 2011/066389, published Jun. 3, 2011 by MedImmune Ltd. et al.
PCT Publication No. WO 2012/145493, published Oct. 26, 2012 by Amplimmune, Inc.
PCT Publication No. WO 2013/013025, published Jan. 24, 2013 by MedImmune Ltd.
PCT Publication No. WO 2013/071068, published May 16, 2013 by Bristol-Myers Squibb Co.
PCT Publication No. WO 2013/079174, published Jun. 6, 2013 by Merck Patent GmbH.
PCT Publication No. WO 2013/173223, published Nov. 21, 2013 by Bristol-Myers Squibb Co.
PCT Publication No. WO 2013/181634, published Dec. 5, 2013 by Sorrento Therapeutics, Inc.
PCT Publication No. WO 2015/019284, published Feb. 12, 2015 by Cambridge Enterprise Ltd.
Pitt L A, Tikhonova A N, Hu H, Trimarchi T, King B et al. (2015) CXCL12-Producing Vascular Endothelial Niches Control Acute T Cell Leukemia Maintenance. Cancer Cell 27(6):755-68.
Reck M, Bondarenko I, Luft A, Serwatowski P, Barlesi F et al. (2013) Ipilimumab in combination with paclitaxel and carboplatin as first-line therapy in extensive-disease-small-cell-lung-cancer: results from a randomized, double-blind, multicenter phase 2 trial. Ann Oncol 24:75-83.
Ribas A (2010) Clinical development of the anti-CTLA-4 antibody tremelimumab. Semin Oncol 37(5):450-4.
Rini B I, Stein M, Shannon P, Eddy S, Tyler A et al. (2011) Phase 1 dose-escalation trial of tremelimumab plus sunitinib in patients with metastatic renal cell carcinoma. Cancer 117:758-67.
Segal N H, Antonia S J, Brahmer J R, Maio M, Blake-Haskins A et al. (2014) Preliminary data from a multi-arm expansion study of MEDI4736, an anti-PD-L1 antibody. J Clin Oncol 32 (suppl. 5S); abstr 3002.
Siegel R, Miller K M, Jemal A (2015) Cancer statistics, 2015. CA Cancer J Clin 65(1):5-29.
Sjoblom T, Jones S, Wood L D, Parsons D W et al. (2006) The consensus coding sequences of human breast and colorectal cancers. Science 314:268-74.
Simon R (1989) Optimal two-stage designs for Phase II clinical trials. Control Clin Trials 10:1-10.
Skolnik J M, Barrett J S, Jayaraman B, Patel D, Adams P C (2007) Shortening the timeline of pediatric Phase 1 trials: The Rolling Six design. J Clin Oncol 26 (2):190-195.
Sorensen M, Pijls-Johannesma M, Felip E (2010) Small-cell lung cancer: ESMO clinical practice guidelines for diagnosis, treatment and follow-up. Ann Oncol (Suppl 5):v120-5.
Spigal D R, Weaver R, McCleod M, Harrid O, Stille J R et al. (2014) Phase 2 study of carboplatin/etoposide plus LY2510924, a CXCR4 peptide antagonist, versus carboplatin/etoposide in patients with extensive stage small cell lung cancer. Ann Oncol 25 (Suppl. 4):iv511-iv516.
Tempero M A, Arnoletti J P, Behrman S W, Ben-Josef E, Benson A B 3rd. (2012)
Pancreatic Adenocarcinoma, version 2.2012: featured updates to the NCCN Guidelines. J Natl Compr Canc Netw 10:703-13.
Topalian S L, Drake C G, Pardoll D M (2012a) Targeting the PD-1/B7-H1(PD-L1) pathway to activate anti-tumor immunity. Curr Opin Immunol 24(2):207-12.
Topalian S L, Hodi F S, Brahmer J R, Gettinger S N et al. (2012b) Safety, activity, and immune correlates of anti-PD-1 antibody in cancer. New Engl J Med 366:2443-54.
Topalian S L, Sznol M, McDermott D F, Kluger H M et al. (2014) Survival, durable tumor remission, and long-term safety in patients with advanced melanoma receiving nivolumab. J Clin Oncol 32(10):1020-30.
U.S. Pat. No. 6,682,736, issued Jan. 27, 2004 to Hanson et al.
U.S. Pat. No. 7,488,802, issued Feb. 10, 2009 to Collins et al.
U.S. Pat. No. 7,892,546, issued Feb. 22, 2011 to Dickerson et al.
U.S. Pat. No. 7,943,743, issued May 17, 2011 to Korman et al.
U.S. Pat. No. 8,008,449, issued Aug. 30, 2011 to Korman et al.
U.S. Pat. No. 8,168,757, issued May 1, 2012 to Finnefrock et al.
U.S. Pat. No. 8,217,149, issued Jul. 10, 2012 to Irving et al.
U.S. Pat. No. 8,354,509, issued Jan. 15, 2013 to Carven et al.
U.S. Pat. No. 8,496,931, issued Jul. 30, 2013 to Pogue et al.
U.S. Publication No. 2015/0037328, published Feb. 5, 2015 by Pfizer, Inc.
Von Hoff D D, Ervin T, Arena F P, Chiorean E G, Infante J et al. (2013) Increased survival in pancreatic cancer with nab-paclitaxel plus gemcitabine. New Engl J Med 369:1691-702.
Wang C, Thudium K B, Han M, Wang X T et al. (2014) In vitro characterization of the anti-PD-1 antibody nivolumab, BMS-936558, and in vivo toxicology in non-human primates. Cancer Imm Res 2(9):846-56.
Wang Z, Ma Q, Li P, Sha H, Li X, Xu J (2013) Aberrant expression of CXCR4 and (3-catenin in pancreatic cancer. Anticancer Res 33(9):4103-10.
Wolchok J D, Weber J S, Maio M, Neyns B, Harmankaya K et al. (2013) Four-year survival rates for patients with metastatic melanoma who received ipilimumab in phase II clinical trials. Ann Oncol 24(8):2174-80.
Yao S, Zhu Y, Chen L (2013) Advances in targeting cell surface signalling molecules for immune modulation. Nature Rev Drug Discov 12:130-46.

1. A method for treating a subject afflicted with a cancer comprising administering to the subject a combination of therapeutically effective amounts of:
(a) an antibody or an antigen-binding portion thereof that binds specifically to Programmed Death-1 (PD-1) or to Programmed Death Ligand-1 (PD-L1); and
(b) an antibody or an antigen-binding portion thereof that binds specifically to C-X-C Chemokine Receptor 4 (CXCR4) or to C-X-C motif chemokine 12 (CXCL12).

2. (canceled)

3. The method of claim 1, wherein the antibody or antigen-binding portion thereof that binds specifically to PD-1 cross-competes with nivolumab for binding to human PD-1.

4-5. (canceled)

6. The method of claim 1, wherein the antibody that binds specifically to PD-1 is nivolumab or pembrolizumab.

7-8. (canceled)

9. The method of claim 1, wherein the antibody or antigen-binding portion thereof that binds specifically to PD-L1 cross-competes with the antibody designated BMS-936559 for binding to human PD-L1.

10-11. (canceled)

12. The method of claim 1, wherein the antibody that binds specifically to PD-L1 is atezolizumab, durvalumab, avelumab, the antibody designated STI-A1014, or the antibody designated BMS-936559.

13-14. (canceled)

15. The method of claim 1, wherein the antibody or antigen-binding portion thereof that binds specifically to CXCR4 cross-competes with ulocuplumab for binding to human CXCR4.

16. (canceled)

17. The method of claim 1, wherein the antibody or antigen-binding portion thereof that binds specifically to CXCR4 comprises a heavy chain constant region which is of a human IgG1, IgG2, IgG3, or IgG4 isotype.

18. The method of claim 17, wherein the antibody or antigen-binding portion thereof that binds specifically to CXCR4 comprises a heavy chain constant region which is of a human IgG1 isotype.

19. (canceled)

20. The method of claim 1, wherein the antibody that binds specifically to CXCR4 is ulocuplumab.

21. The method of claim 1, wherein the antibody that binds specifically to CXCR4 is a human IgG1 variant of ulocuplumab.

22-24. (canceled)

25. The method of claim 1, wherein the anti-CXCL12 antibody or antigen-binding portion thereof that binds to CXCL12 binds to the same epitope region of CXCL12a as does the antibody designated 2A5 or the antibody designated 1H2.

26-27. (canceled)

28. The method of claim 1, wherein the antibody that binds to CXCL12 is the antibody designated 2A5 or the antibody designated 1H2.

29. (canceled)

30. The method of claim 1, wherein the cancer is a solid tumor.

31. (canceled)

32. The method of claim 30, wherein the solid tumor is a cancer selected from pancreatic cancer (PAC), small cell lung cancer (SCLC), hepatocellular carcinoma (HCC), squamous cell carcinoma, non-small cell lung cancer, squamous non-small cell lung cancer (NSCLC), non-squamous NSCLC, glioma, gastrointestinal cancer, renal cancer, ovarian cancer, liver cancer, colorectal cancer, endometrial cancer, kidney cancer, prostate cancer, thyroid cancer, neuroblastoma, glioblastoma, stomach cancer, bladder cancer, hepatoma, breast cancer, colon carcinoma, head and neck cancer, gastric cancer, germ cell tumor, pediatric sarcoma, sinonasal natural killer, melanoma, skin cancer, bone cancer, cervical cancer, uterine cancer, carcinoma of the fallopian tubes, carcinoma of the endometrium, carcinoma of the cervix, carcinoma of the vagina, carcinoma of the vulva, cancer of the anal region, testicular cancer, cancer of the esophagus, cancer of the small intestine, cancer of the endocrine system, cancer of the parathyroid gland, cancer of the adrenal gland, sarcoma of soft tissue, cancer of the urethra, cancer of the ureter, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, and cancers of viral origin.

33. The method of claim 1, wherein the cancer is a hematological malignancy.

34. The method of claim 33, wherein the hematological malignancy is selected from acute lymphoblastic leukemia (ALL), acute myelogenous leukemia (AML), chronic lymphocytic leukemia (CLL), chronic myelogenous leukemia (CML), Hodgkin's lymphoma (HL), non-Hodgkin's lymphomas (NHLs), multiple myeloma, smoldering myeloma, monoclonal gammopathy of undetermined significance (MGUS), advanced, metastatic, refractory and/or recurrent hematological malignancies, and any combinations of said hematological malignancies.

35-47. (canceled)

48. The method of claim 1, comprising administering to the subject a combination of:
(a) an antibody or an antigen-binding portion thereof that binds specifically to PD-1 and inhibits PD-1/PD-L1 signaling, wherein the anti-PD-1 antibody or portion thereof is administered at a dose of about 2 or about 3 mg/kg body weight once every 2 or 3 weeks; and
(b) an antibody or an antigen-binding portion thereof that binds specifically to CXCR4 and inhibits CXCR4/CXCL12 signaling, wherein the anti-CXCR4 antibody or portion thereof is administered at a flat dose of about 200, about 400 or about 800 mg weekly.

49. The method of claim 1, wherein the antibody that binds specifically to CXCR4 is an antibody comprising an Fc region that mediates effector functions.

50-62. (canceled)

63. A method for reducing adverse events in a subject undergoing treatment for cancer comprising administering to the subject a combination of:
(a) an antibody or an antigen-binding portion thereof that binds specifically to Programmed Death-1 (PD-1) or to Programmed Death Ligand-1 (PD-L1); and
(b) an antibody or an antigen-binding portion thereof that binds specifically to C-X-C Chemokine Receptor 4 (CXCR4) or to C-X-C motif chemokine 12 (CXCL12),
wherein at least one of the antibodies or portions thereof is administered at a subtherapeutic dose.

64-65. (canceled)

66. A kit for treating a subject afflicted with a cancer, the kit comprising:
(a) one or more dosages ranging from about 0.1 to about 20 mg/kg body weight of an antibody or an antigen-binding portion thereof that binds specifically to PD-1 or to PD-L1;
(b) one or more dosages ranging from about 200 to about 1600 mg of an antibody or an antigen-binding portion thereof that binds specifically to CXCR4 or to CXCL12; and
(c) instructions for using the antibody or portion thereof that binds specifically to PD-1 or to PD-L1 and the antibody or portion thereof that binds specifically to CXCR4 or to CXCL12 in the method of claim 1.


SEQUENCE LISTING (18 sequences)

SEQ ID NO: 1 - protein, 113 aa, Homo Sapien [sequence omitted]

SEQ ID NO: 2 - protein, 107 aa, Homo Sapien [sequence omitted]

SEQ ID NO: 3 - protein, 439 aa, Homo Sapien
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 214 aa, Homo Sapien
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 123 aa, Homo Sapien
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 6 - protein, 106 aa, Homo Sapien
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 7 - protein, 449 aa, Homo Sapien
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 8 - protein, 213 aa, Homo Sapien
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 125 aa, Homo Sapien
EVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSS

SEQ ID NO: 10 - protein, 107 aa, Homo Sapien
DIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIK

SEQ ID NO: 11 - protein, 451 aa, Homo Sapien
EVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 214 aa, Homo Sapien
DIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 454 aa, Homo Sapien
EVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 501 aa, Homo Sapien
EVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKGLPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 15 - protein, 124 aa, Homo Sapien
EVQLVESGGGLVQPGGSLRLSCAASGFTFSSYWMSWVRQAPGKGLEWVANMNQDGSEKYYVDSVKGRFTISRDNAKNSLYLQMNSLRAEDTAVYYCARDLTGPYYYDYYGMDVWGQGTTVTVSS

SEQ ID NO: 16 - protein, 107 aa, Homo Sapien
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGQGTKVEIK

SEQ ID NO: 17 - protein, 450 aa, Homo Sapien
EVQLVESGGGLVQPGGSLRLSCAASGFTFSSYWMSWVRQAPGKGLEWVANMNQDGSEKYYVDSVKGRFTISRDNAKNSLYLQMNSLRAEDTAVYYCARDLTGPYYYDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 214 aa, Homo Sapien
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC